(19) United States
(12) Patent Application Publication
(10) Pub. No.: US 2026/0224580 A1
(43) Pub. Date: Aug. 6, 2026

(54) ORAL PRODUCT

(71) Applicant: NICOVENTURES TRADING LIMITED, London (GB)

(72) Inventors: Deborah SMITH, London (GB); Aideen DALY, London (GB); Jessica MUSHONGANONO, London (GB); Anna AZZOPARDI, London (GB); Jason ADAMSON, London (GB); Charlotte CADDICK, London (GB); Olivia O'SHEA, London (GB)

(21) Appl. No.: 19/118,394

(22) PCT Filed: Oct. 6, 2023
(86) PCT No.: PCT/GB2023/052589

§ 371 Date: Apr. 4, 2025

(30) Foreign Application Priority Data
Oct. 7, 2022 (GB) 2214803.5

(51) Int. Cl. A61K 31/522; A61K 9/00; A61K 31/131; A61K 31/375; A61K 31/4188; A61K 31/4415; A61K 31/51; A61K 31/525; A61K 36/258; A61P 25/28

(52) U.S. Cl. CPC A61K 31/522; A61K 9/0095; A61K 31/131; A61K 31/375; A61K 31/4188; A61K 31/4415; A61K 31/51; A61K 31/525; A61K 36/258; A61P 25/28

(57) ABSTRACT

The invention relates to an oral pouch for placement in the oral vestibule for absorbing an active ingredient composition by a human, comprising a pouch sleeve made of a material which is permeable to saliva and an active ingredient composition containing caffeine and taurine.

### FIELD

The present disclosure relates to an oral product, a process for producing the oral product, as well as to uses of said oral product.

### BACKGROUND

The present disclosure relates to products and compositions intended for human use. The products are configured for oral use and deliver substances such as flavours and/or active ingredients during use.

Products that are easy to administer orally and which can provide a beneficial effect on particular mood states of a human or animal have become popular in recent years. For example, confectionary-type products (e.g. gummies or pastilles) containing vitamins or other mood-enhancing actives provide a convenient and pleasant mode of administration for such active ingredients. Other convenient modes of administration are food and beverages, for example energy drinks. Such products may include active ingredients that are delivered to the user in order to cause a biological response in the user that may enhance physical or mental performance of the user.

Beverages containing theanine and caffeine have been described in U.S. Pat. Nos. 5,780,086 and 6,268,009. More recently, EP1819241 describes beverages containing theanine and caffeine in a ratio of from 5:1 to 1:1.5. Other beverages, such as chamomile tea and other herbal teas, are consumed by some users to aid sleep and relaxation.

It would be desirable to provide an oral product configured for oral use which may deliver active ingredients to the consumer in an enjoyable and effective form, such as in the form of liquid shots.

### BRIEF SUMMARY

The present disclosure generally provides products configured for oral use, the products comprising a combination of active ingredients. The oral products may be in any form suitable for oral use, such as in tablet or lozenge form, as a loose powder, or liquid form (e.g. as a beverage, such as a shot).

In accordance with some embodiments described herein, there is provided an oral product comprising a combination of active ingredients, wherein the combination comprises: (i) caffeine; (ii) a combination of B vitamins comprising at least vitamin B2, vitamin B3, vitamin B6, vitamin B9 and vitamin B12; and (iii) taurine.

In accordance with other embodiments described herein, there is provided an oral product comprising a combination of active ingredients, wherein the combination comprises: (i) caffeine; (ii) a combination of B vitamins comprising at least vitamin B1, vitamin B2, vitamin B6, and vitamin B7; and (iii) taurine.

In accordance with some embodiments described herein, there is provided a process for preparing an oral product, the process comprising:
(a) combining active ingredients, wherein the active ingredients include: (i) caffeine; (ii) a combination of B vitamins comprising at least vitamin B2, vitamin B3, vitamin B6, vitamin B9 and vitamin B12; and (iii) taurine,
(b) contacting the active ingredients with water, and
(c) mixing the active ingredients with water to prepare an oral product.

In accordance with other embodiments described herein, there is provided a process for preparing an oral product, the process comprising:
(a) combining active ingredients, wherein the active ingredients include: (i) caffeine; (ii) a combination of B vitamins comprising at least vitamin B1, vitamin B2, vitamin B6, and vitamin B7; and (iii) taurine,
(b) contacting the active ingredients with water, and
(c) mixing the active ingredients with water to prepare an oral product.

In accordance with some embodiments described herein, there is provided a use of a combination of active ingredients to increase alertness and/or energy levels of a human or animal, wherein the combination of active ingredients comprises (i) caffeine; (ii) a combination of B vitamins comprising at least vitamin B2, vitamin B3, vitamin B6, vitamin B9 and vitamin B12; and (iii) taurine.

In accordance with other embodiments described herein, there is provided a use of the combination of active ingredients to increase alertness and/or energy levels of a human or animal, wherein the combination of active ingredients comprises (i) caffeine; (ii) a combination of B vitamins comprising at least vitamin B1, vitamin B2, vitamin B6, and vitamin B7; and (iii) taurine.

In accordance with some embodiments described herein, there is provided a use of a combination of active ingredients to increase mental and/or physical energy of a human or animal, wherein the combination of active ingredients comprises (i) caffeine; (ii) a combination of B vitamins comprising at least vitamin B2, vitamin B3, vitamin B6, vitamin B9 and vitamin B12; and (iii) taurine.

In accordance with other embodiments described herein, there is provided a use of a combination of active ingredients to increase mental and/or physical energy of a human or animal, wherein the combination of active ingredients comprises (i) caffeine; (ii) a combination of B vitamins comprising at least vitamin B1, vitamin B2, vitamin B6, and vitamin B7; and (iii) taurine.

In accordance with some embodiments described herein, there is provided a use of a combination of active ingredients to increase the cognitive performance of a human or animal, wherein the combination of active ingredients comprises (i) caffeine; (ii) a combination of B vitamins comprising at least vitamin B1, vitamin B2, vitamin B6, and vitamin B7; and (iii) ginseng.

In accordance with some embodiments described herein, there is provided an oral product comprising a combination of active ingredients, wherein the combination of active ingredients comprises (i) caffeine; (ii) ginseng and (iii) taurine.

In accordance with some embodiments described herein, there is provided an oral product in liquid form comprising a combination of active ingredients, wherein the combination of active ingredients comprises: (i) caffeine; (ii) a combination of B vitamins comprising vitamin B2, vitamin B3, vitamin B6, vitamin B9 and vitamin B12; and (iii) ginseng.

In accordance with some embodiments described herein, there is provided an oral product in liquid form comprising a combination of active ingredients, wherein the combination of active ingredients comprises: (i) caffeine; (ii) a combination of B vitamins comprising vitamin B1, vitamin B2, vitamin B6, and vitamin B7; and (iii) ginseng.

In accordance with some embodiments described herein, there is provided an oral product comprising a combination of active ingredients, wherein the combination of active ingredients comprises: (i) caffeine; (ii) at least one B vitamin selected from the group consisting of vitamin B1, vitamin B2, vitamin B3, vitamin B5, vitamin B6, vitamin B7, vitamin B9 and vitamin B12; (iii) taurine; and (iv) vitamin C.

In preferred embodiments, each of the oral products of the above embodiments may be provided in the form of a liquid dosage form, such as a shot (i.e. a liquid having a volume of up to about 100 mL).

In accordance with some embodiments described herein there is provided a use of a combination of active ingredients to increase the cognitive performance of a human or animal, wherein the combination of active ingredients comprises (i) caffeine; (ii) a combination of B vitamins comprising at least vitamin B1, vitamin B2, vitamin B6, and vitamin B7; and (iii) taurine.

In accordance with some embodiments described herein there is provided a use of a combination of active ingredients to increase the cognitive performance of a human or animal, wherein the combination of active ingredients comprises (i) caffeine; (ii) taurine; and (iii) ginseng.

These and other features, aspects, and advantages of the disclosure will be apparent from a reading of the following detailed description. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

For ease of reference, these and further aspects of the present invention are now discussed under appropriate section headings. However, the teachings under each section are not necessarily limited to each particular section.

### BRIEF DESCRIPTION OF THE DRAWINGS

Having thus described aspects of the disclosure in the foregoing general terms, reference will now be made to the accompanying drawings, which are not necessarily drawn to scale. The drawings are exemplary only, and should not be construed as limiting the disclosure.

Embodiments of the invention will now be described, by way of example only, with reference to accompanying drawings, in which:

FIG. 1 is a graph showing the results of testing for cognitive accuracy (RVIP) after administration of an oral product of Example 3 compared with placebo.

FIG. 2 is a graph showing the results for mental arithmetic (serial subtraction −3s) after administration of an oral product of Example 3 compared with placebo.

FIG. 3 is a graph showing alertness measured by VAS after administration of an oral product of Example 3 compared with placebo.

FIG. 4 is a graph showing physical energy measured by VAS after administration of an oral product of Example 3 compared with placebo.

FIG. 5 is a graph showing the result of testing for mental fatigue measured by POMS, comparing a placebo with Example 3.

FIG. 6 is a graph showing the result of testing for cognitive accuracy (RVIP), comparing a placebo and 5-Hour Energy with Example 3.

FIG. 7 is a graph showing results of mental arithmetic (serial subtraction (−3)) after administration of an oral product of Example 3 compared with placebo and 5-Hour Energy.

FIG. 8 is a graph showing alertness measured by VAS after administration of an oral product of Example 3 compared with placebo and 5-Hours Energy.

FIG. 9 is a graph showing the physical energy measured by VAS after administration of an oral product of Example 3 compared with placebo and 5-Hour Energy.

FIG. 10 is a graph showing cognitive accuracy over time (post-consumption of the oral product) measured by RVIP for an oral product of Example 7 compared with placebo. An asterisk (*) indicates a significant change from placebo.

FIG. 11 is a graph showing memory recall over time measured by NWM (p=0.026) after consumption of an oral product of Example 7 compared with placebo.

FIG. 12 is a graph showing mental arithmetic performance measured by serial subtraction −3's (COMPASS) (p<0.001) after consumption of an oral product of Example 7 compared with placebo.

FIG. 13 is a graph showing cognitive accuracy measured by RVIP (p<0.001) after consumption of an oral product of Example 7 compared with placebo.

FIG. 14 is a graph showing mental processing measured by RVIP (p=0.037) after consumption of an oral product of Example 7 compared with placebo.

FIG. 15 is a graph showing mental arithmetic performance measured by serial subtraction −7's (COMPASS) (p<0.001) after consumption of an oral product of Example 7 compared with placebo.

FIG. 16 is a graph showing global cognition (z-score) (p<0.001) based on the measures of FIGS. 11 to 15 after consumption of an oral product of Example 7 compared with placebo. A z-score is a standard score indicating how many standard deviations an individual value is away from the mean and allow the comparison of scores from different tests. Z-scores were first calculated based on the non-adjusted raw data for each test. The respective z-scores were then combined (e.g. for RVIP, serial subtraction −3's and −7's). Lastly, scores were computed for each time period and adjusted by baseline subtraction.

FIG. 17 is a graph showing mental arithmetic performance over time measured by serial subtraction −3's (COMPASS) after consumption of an oral product of Example 7 compared with placebo. An asterisk (*) indicates a significant change from placebo.

FIG. 18 is a graph showing mental arithmetic performance over time measured by serial subtraction −7's (COMPASS) after consumption of an oral product of Example 7 compared with placebo. An asterisk (*) indicates a significant change from placebo.

FIG. 19 is a graph showing perceived alertness measured by VAS (p<0.001) after consumption of an oral product of Example 7 compared with placebo.

FIG. 20 is a graph showing perceived physical energy measured by VAS (p=0.001) after consumption of an oral product of Example 7 compared with placebo.

FIG. 21 is a graph showing perceived fatigue reduction measured by VAS (p<0.001) after consumption of an oral product of Example 7 compared with placebo.

FIG. 22 is a graph showing perceived feeling of stimulation measured by VAS (p<0.001) after consumption of an oral product of Example 7 compared with placebo.

FIG. 23 is a graph showing perceived reduction of vigour measured by VAS (p=0.043) after consumption of an oral product of Example 7 compared with placebo.

FIG. 24 is a graph showing perceived alertness over time measured by VAS after consumption of an oral product of Example 7 compared with placebo. An asterisk (*) indicates a significant change from placebo.

FIG. 25 is a graph showing perceived physical energy over time measured by VAS after consumption of an oral product of Example 7 compared with placebo. An asterisk (*) indicates a significant change from placebo.

### DETAILED DESCRIPTION

It is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

As used in this specification and the claims, the singular forms “a,” “an,” and “the” include plural referents unless the context clearly dictates otherwise. Reference to “dry % by weight” or “dry weight basis” refers to weight on the basis of dry ingredients (i.e., all ingredients except water). Reference to “wet weight” refers to the weight of the product or composition including water. Unless otherwise indicated, reference to “% by weight” (or “% by weight”) of a product or composition reflects the total wet weight of the product or composition (i.e., including water).

In this specification, unless otherwise stated, the term “about” modifying the quantity of an ingredient in the oral product of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the oral product, or to carry out the methods; and the like. The term “about” also encompasses amounts that differ due to different equilibrium conditions for a product or composition resulting from a particular initial mixture. Whether or not modified by the term “about”, the claims include equivalents to the quantities.

### Oral Product

As described herein, there is provided an oral product comprising a combination of active ingredients, wherein the combination comprises: (i) caffeine; (ii) a combination of B vitamins comprising at least vitamin B2, vitamin B3, vitamin B6, vitamin B9 and vitamin B12; and (iii) taurine.

Also described herein, there is provided an oral product comprising a combination of active ingredients, wherein the combination comprises: (i) caffeine; (ii) a combination of B vitamins comprising at least vitamin B1, vitamin B2, vitamin B6, and vitamin B7; and (iii) taurine.

The oral product is configured for oral use, and thus for insertion into the user's mouth (i.e. oral cavity). In this specification, unless otherwise stated, the term “oral” in connection to a product refers to a product which, in normal use, is suited to be ingested or placed somewhere in the oral cavity of the user. For example, the product may be in the form of a liquid that may be consumed orally by the user (i.e. in the form of a beverage), or the product may be placed in the mouth.

The ranges provided herein provide preferred amounts of each of the components. Each of these ranges may be taken alone or combined with one or more other component ranges to provide a preferred aspect of the invention.

### Combination of Active Ingredients

The product as disclosed herein includes active ingredients.

The active substance as used herein may be a physiologically active material, which is a material intended to achieve or enhance a physiological response. The active substance may for example be selected from nutraceuticals, nootropics, psychoactives. The active substance may be naturally occurring or synthetically obtained.

The combination of active ingredients comprises suitable active ingredients that cause a biological response in a human or animal. The active ingredient as used herein may be a physiologically active material, which is a material intended to achieve or enhance a physiological response.

According to one aspect of the invention, the combination of active ingredients comprises at least (i) caffeine; (ii) a combination of B vitamins comprising at least vitamin B2, vitamin B3, vitamin B6, vitamin B9 and vitamin B12; and (iii) taurine.

According to another aspect of the invention, the combination of active ingredients comprises at least (i) caffeine; (ii) a combination of B vitamins comprising at least vitamin B1, vitamin B2, vitamin B6, and vitamin B7; and (iii) taurine.

Each of the active ingredients may be present in an amount suitable to provide the desired biological response in a human animal. The specific combination of active ingredients included in the oral product has been found by the inventors to provide a rapid onset of mental and/or physical energy. The combination of active ingredients has been found in particular to provide a short, medium or long-term mental and/or physical energy boost for the user. In particular, the specific combination of active ingredients in the oral product has been found to increase mental energy (i.e. improve cognitive performance, increase energy (alertness, arousal) or reduce mental fatigue and to increase motivation of the consumer to perform mental tasks) and to increase physical energy (i.e. improve endurance performance and capacity). The present inventors have found that the specific combination of active ingredients of the present invention may improve energy levels over a short or sustained period of time. The increase in energy levels may also increase the user's overall feelings of enjoyment and enthusiasm, with feelings of confidence, motivation, pleasure and excitement being increased. The inventors have also found that there is an improvement in cognitive performance for at least 90 minutes post-consumption.

It has also been found that the combination of active ingredients described herein may provide a safe product, with desirable pharmacokinetics (Tmax, Cmax, half life), bioavailability and metabolism.

Caffeine is a central nervous system stimulant. Caffeine (1,3,7-trimethylxanthine) exists as a bitter substance, an alkaloid which is naturally found in a variety of plant species, including tea leaves, kola nuts, cocoa, guarana, and coffee beans. Once ingested, caffeine is metabolised through the liver via the cytochrome p450 enzyme system and is capable of entering all body tissues, as well as crossing the blood brain barrier. Caffeine may be used to enhance alertness, attention, cognitive performance as well as physical performance.

The caffeine may be included as a natural stimulant, meaning that it is naturally derived. By “naturally derived” is meant that the caffeine is in a purified form, outside its natural (e.g. botanical) matrix. For example, caffeine may be obtained by extraction and purification from botanical sources (e.g. tea). Alternatively or in addition, the caffeine may be provided in synthetic form; i.e. obtained by chemical synthesis. The caffeine may be provided in the form of an extract or in the form of a powder (e.g. a powder extract). For example, the caffeine may be included in the form of a green tea powder extract, black tea powder extract. The caffeine may also be provided in the form of guarana seed powder extract. In some embodiments, the caffeine is present in an encapsulated form. An example of an encapsulated caffeine is Vitashure®, available from Balchem Corp., 52 Sunrise Park Road, New Hampton, NY, 10958.

The skilled person will appreciate that where caffeine is naturally derived, e.g. by extraction and purification from botanical sources, and such naturally derived caffeine includes one or more B vitamins, said B vitamins are in addition to those B vitamins explicitly recited for the oral product of the present invention.

The caffeine may be present in any suitable amount, such as in an amount of at least about 0.001% by weight, at least about 0.01% by weight, or at least about 0.1% by weight of the oral product. In some embodiments, the caffeine may be present in an amount of no greater than about 20% by weight, no greater than about 10% by weight, no greater than about 5% by weight, or no greater than about 1% by weight of the oral product.

The caffeine may be present in an amount of from about 0.001% to about 20% by weight of the oral product. In some embodiments, the caffeine is present in an amount of from about 0.01% to about 10% by weight of the oral product. The caffeine may preferably be present in an amount of from about 0.1% to about 5% by weight of the oral product.

In some embodiments, the caffeine is present in an amount of from about 0.01% to about 4% by weight of the oral product, such as from about 0.05% to about 3% by weight of the oral product, such as from about 0.1% to about 2.5% by weight of the oral product, such as from about 0.15% to about 2% by weight of the oral product, such as from about 0.2% to about 1.5% by weight of the oral product, such as from about 0.25% to about 1% by weight of the oral product. In some preferred embodiments, the caffeine may be present in an amount of from about 0.1% to about 1% by weight of the oral product.

The combination of active ingredients further comprises a combination of B vitamins comprising at least vitamin B2, vitamin B3, vitamin B6, vitamin B9 and vitamin B12. Alternatively, the combination of active ingredients further comprises a combination of B vitamins comprising vitamin B1, vitamin B2, vitamin B6, and vitamin B7.

The term “B vitamins” includes a group of eight water-soluble vitamins that have effect in cellular functioning; e.g. cell metabolism and synthesis of red blood cells. B vitamins act as coenzymes in a substantial proportion of the enzymatic processes for cellular physiological functioning. The B vitamins may generally be divided into two categories: those that act as co-enzymes for catabolic enzymatic reactions (leading to generation of energy) and those that act as co-enzymes for anabolic enzymatic reactions (leading to enhanced brain function).

B vitamins are considered to have roles in aspects of brain function and energy production. The eight water soluble B vitamins are vitamin B1 (thiamin), vitamin B2 (riboflavin), vitamin B3 (niacin), vitamin B5 (panthothenic acid or salt thereof, e.g. calcium pantothenate), vitamin B6 (pyridoxine hydrochloride), vitamin B7 (biotin), vitamin B9 (folate or folic acid), vitamin B12 (cyanocobalamin). Vitamins B2, B3, B6, B9 and B12 have been found to support anabolic metabolism and to boost brain function. Vitamins B1, B2, B3, B5 and B7 have been found to support catabolic metabolism and to boost energy levels.

In preferred embodiments of one aspect, the oral product includes all five B vitamins that support anabolic metabolism. In other words, according to a first aspect, the combination of active ingredients includes a combination of vitamins B2, B3, B6, B9 and B12. It has been found that the inclusion of at least these B vitamins may reduce perceived physical and mental fatigue as well as enhance psychological and cognitive functions. Each of these B vitamins may contribute to the reduction of tiredness and fatigue, and the inclusion of all five B vitamins may lead to a synergistic effect in this regard.

In some embodiments, the combination of B vitamins consists essentially of all anabolic vitamins; i.e. a combination of vitamins B2, B3, B6, B9 and B12. In some embodiments, the combination of B vitamins consists of all anabolic vitamins; i.e. a combination of vitamins B2, B3, B6, B9 and B12. The term “anabolic B vitamin” refers to those B vitamins that have an effect on anabolic pathways, while the term “catabolic B vitamin” refers to those B vitamins that have an effect on catabolic pathways.

In some embodiments, the combination of B vitamins further comprises at least one catabolic B vitamin selected from the group consisting of vitamin B1, vitamin B5, vitamin B7 and combinations thereof.

In any such embodiments, the ratio of the total amount of anabolic B vitamins (i.e. vitamins B2, B3, B6, B9 and B12) to the total amount of catabolic B vitamins (i.e. vitamins B1, B5 and B7) may be from about 5:1 to about 1:5, preferably from about 2:1 to about 1:2 and more preferably from about 2:1 to about 1:1.

In preferred embodiments of another aspect, the oral product includes a mixture of B vitamins that support anabolic and catabolic metabolism. This mixture may be advantageous for stability purposes, e.g. have a longer shelf-life in the oral product compared to a product with all eight B vitamins. In such embodiments, the oral product comprises at least vitamin B1, vitamin B2, vitamin B6, and vitamin B7.

In some embodiments, the combination of B vitamins further comprises one or more of vitamin B3, vitamin B5, vitamin B9, or vitamin B12.

In some embodiments, the combination of B vitamins consists essentially of vitamin B1, vitamin B2, vitamin B6 and vitamin B7. Preferably, the combination of B vitamins consists of vitamin B1, vitamin B2, vitamin B6, and vitamin B7. As noted above, the skilled person will appreciate that where caffeine is naturally derived, e.g. by extraction and purification from botanical sources, and such naturally derived caffeine includes one or more B vitamins, said B vitamins are in addition to those B vitamins explicitly recited for the oral product of the present invention.

In any such embodiments, the ratio of the total amount of anabolic B vitamins (i.e. vitamins B2, B3, B6, B9 and B12) to the total amount of catabolic B vitamins (i.e. vitamins B1, B5 and B7) may be from about 10:1 to about 1:10, preferably from about 5:1 to about 1:5 and more preferably from about 5:1 to about 1:1.

In any such embodiments, the ratio of the total amount of anabolic B vitamins (i.e. vitamins B2 and B6) to the total amount of catabolic B vitamins (i.e. vitamins B1 and B7) may be from about 10:1 to about 1:10, preferably from about 5:1 to about 1:5 and more preferably from about 5:1 to about 1:1.

The amounts of the B vitamins included in the composition may be any amount suitable to provide the desired effect while also providing a product that is safe to consume and has reduced side effects.

The total amount of B vitamins included in the combination may be any suitable amount to provide the desired effect of boosting brain function. In some embodiments, the total amount of anabolic B vitamins (i.e. the total amount of the combination of vitamins B2, B3, B6, B9 and/or B12) may be at least about 0.0001% by weight, preferably at least about 0.001% by weight, and more preferably at least about 0.01% by weight of the oral product. For example, the total amount of anabolic B vitamins may be from about 0.0001% to about 5% by weight of the oral product, and preferably from about 0.001% to about 1%. For example, this amount may be from about 0.01% to about 0.5% by weight of the oral product, or from about 0.05% to about 0.5% by weight of the oral product.

Where present, the total amount of catabolic B vitamins (i.e. the total amount of the combination of vitamins B1, B5 and/or B7) may be at least about 0.0001% by weight, preferably at least about 0.001% by weight, and more preferably at least about 0.01% by weight of the oral product. For example, the total amount of catabolic B vitamins (e.g. where the catabolic B vitamins are vitamin B1, vitamin B2, vitamin B3, vitamin B5 and/or vitamin B7) may be from about 0.0001% to about 5% by weight of the oral product, and preferably from about 0.001% to about 1%. For example, this amount may be from about 0.01% to about 0.5% by weight of the oral product.

The total amount of B vitamins included in the combination may be any suitable amount to provide the desired effect of boosting brain function. In some embodiments, the total amount of B vitamins is from about 0.001% to about 5% by weight, and preferably from about 0.001% to about 1% by weight of the oral product. In some embodiments, the total amount of B vitamins may be from about 0.01% to about 4% by weight, such as from about 0.025% to about 3% by weight, such as from about 0.05% to about 2% by weight of the oral product, and preferably from about 0.05% to about 1% by weight of the oral product.

In some embodiments, vitamin B2 may be present in an amount of from about 1% to about 20% by weight of the total amount of B vitamin in the oral product. For example, vitamin B2 may be present in an amount of from about 5% to about 15% by weight of the total amount of B vitamin in the oral product.

In the oral product containing at least vitamins B1, B2, B6 and B7, vitamin B2 may be present in an amount of from about 10% to about 50% by weight of the total amount of B vitamin in the oral product. In preferred embodiments, vitamin B2 may be present in an amount of from about 15% to about 35% by weight of the total amount of B vitamin in the oral product.

In some embodiments, vitamin B3 may be present in an amount of from about 20% to about 80% by weight of the total amount of B vitamin in the oral product. For example, vitamin B3 may be present in an amount of from about 40% to about 70% by weight, and preferably from about 45% to about 60% by weight of the total amount of B vitamin in the oral product.

In the oral product containing at least vitamins B1, B2, B6 and B7, the oral product may be substantially free of (i.e. include no more than 0.0001% by weight of) vitamin B3, or may be free of (i.e. include 0%) vitamin B3.

In some embodiments, vitamin B6 may be present in an amount of from about 1% to about 15% by weight of the total amount of B vitamin in the oral product. For example, vitamin B6 may be present in an amount of from about 5% to about 10% by weight of the total amount of B vitamin in the oral product.

In the oral product containing at least vitamins B1, B2, B6 and B7, vitamin B6 may be present in an amount of from about 10% to about 75% by weight of the total amount of B vitamin in the oral product. In preferred embodiments, vitamin B6 may be present in an amount of from about 15% to about 65% by weight of the total amount of B vitamin in the oral product.

In some embodiments, vitamin B9 may be present in an amount of from about 0.1% to about 10% by weight of the total amount of B vitamin in the oral product. For example, vitamin B9 may be present in an amount of from about 1% to about 5% by weight of the total amount of B vitamin in the oral product.

In the oral product containing at least vitamins B1, B2, B6 and B7, the oral product may be substantially free of (i.e. include no more than 0.0001% by weight of) vitamin B9, or may be free of (i.e. include 0%) vitamin B9.

In some embodiments, vitamin B12 may be present in an amount of from about 0.01% to about 30% by weight, such as from about 0.1% to about 25% by weight, such as from about 1% to about 20% by weight of the total amount of B vitamin in the oral product. For example, vitamin B12 may be present in an amount of from about 5% to about 15% by weight of the total amount of B vitamin in the oral product.

In the oral product containing at least vitamins B1, B2, B6 and B7, the oral product may be substantially free of (i.e. include no more than 0.0001% by weight of) vitamin B12, or may be free of (i.e. include 0%) vitamin B12.

Where present, vitamin B1 may be present in an amount of from about 1% to about 10% by weight of the total amount of B vitamin in the oral product. For example, where present, vitamin B1 may be present in an amount of from about 2.5% to about 7.5% by weight of the total amount of B vitamin in the oral product. In some embodiments, the oral product may be substantially free of (i.e. include no more than 0.0001% by weight of) vitamin B1, or may be free of (i.e. include 0%) vitamin B1.

In the oral product containing at least vitamins B1, B2, B6 and B7, vitamin B1 may be present in an amount of from about 1% to about 45% by weight of the total amount of B vitamin in the oral product. In preferred embodiments, vitamin B1 may be present in an amount of from about 5% to about 40% by weight of the total amount of B vitamin in the oral product.

Where present, vitamin B5 may be present in an amount of from about 1% to about 20% by weight of the total amount of B vitamin in the oral product. For example, where present, vitamin B5 may be present in an amount of from about 5% to about 15% by weight of the total amount of B vitamin in the oral product. In some embodiments, the oral product may be substantially free of vitamin B5, or may be free of vitamin B5.

In the oral product containing at least vitamins B1, B2, B6 and B7, the oral product may be substantially free of (i.e. include no more than 0.0001% by weight of) vitamin B5, or may be free of (i.e. include 0%) vitamin B5.

Where present, vitamin B7 may be present in an amount of from about 0.01% to about 5% by weight of the total amount of B vitamin in the oral product. For example, where present, vitamin B7 may be present in an amount of from about 0.01% to about 1% by weight of the total amount of B vitamin in the oral product. In some embodiments, the oral product may be substantially free of vitamin B7, or may be free of vitamin B7.

In the oral product containing at least vitamins B1, B2, B6 and B7, vitamin B7 may be present in an amount of from about 0.01% to about 20% by weight of the total amount of B vitamin in the oral product. In preferred embodiments, vitamin B7 may be present in an amount of from about 0.1% to about 15% by weight of the total amount of B vitamin in the oral product.

Taurine or 2-aminoethanesulfonic acid is an organic compound widely distributed in animal tissues. Taurine is known to play a role in energy metabolism. The combination of taurine with caffeine may lead to taurine neutralising some of the negative side effects that excess caffeine may cause (e.g. the jitters or increased heart rate). The inclusion of taurine may improve muscle strength and endurance, and performance during physical activity. The addition of taurine may also enhance energy metabolism.

The taurine may be present in any suitable amount, such as in an amount of at least about 0.001% by weight, at least about 0.01% by weight, or at least about 0.1% by weight of the oral product. In some embodiments, the taurine may be present in an amount of no greater than about 20% by weight, no greater than about 10% by weight, or no greater than about 5% by weight of the oral product.

The taurine may be present in an amount of from about 0.001% to about 20% by weight of the oral product. In some embodiments, the taurine is present in an amount of from about 0.01% to about 10% by weight of the oral product. The taurine may preferably be present in an amount of from about 0.05% to about 5% by weight of the oral product, or from about 0.05% to about 1% by weight of the oral product.

In some preferred embodiments, the taurine is present in an amount of from about 0.01% to about 0.1% by weight of the oral product. In other preferred embodiments, the taurine is present in an amount of from about 0.05% to about 3% by weight of the oral product, such as from about 0.1% to about 2.5% by weight of the oral product, such as from about 0.2% to about 1% by weight of the oral product, such as from about 0.3% to about 0.5% by weight of the oral product.

In some embodiments, the taurine is present in an amount of from about 0.01% to about 0.5% by weight of the oral product.

In addition to caffeine, a combination of B vitamins, and taurine, the combination of active ingredients may include one or more additional active ingredients. The additional active ingredients may be any suitable active ingredient that increase physical and/or mental energy levels and/or increase alertness, and may for example be selected from nutraceuticals, nootropics, psycho-actives. The additional active ingredient may be naturally occurring or synthetically obtained.

Non-limiting examples of additional active ingredients include those falling in the categories of botanical ingredients (e.g. ginseng, Bacopa monieri or Rhodiola rosea, or the like), stimulants, amino acids or analogues (e.g. L-tyrosine, L-theanine), and/or nutraceutical, and/or medicinal ingredients (e.g., further vitamins, such as A, C, D, E or K, and/or cannabinoids, such as tetrahydrocannabinol (THC) and cannabidiol (CBD)).

The additional active ingredient may comprise for example L-tyrosine, L-theanine, theine, vitamins such as vitamin A, vitamin C, vitamin D, vitamin E, vitamin K, ginseng, Echinacea, cannabinoids, choline, alpha GPC, theobromine, Guyasa, maca, matcha tea, methylliberine, nitrate or beetroot juice, yerba mate, glucose, theacrine, or constituents, derivatives, or combinations thereof.

In some embodiments, the one or more additional active ingredients is selected from a botanical ingredient (e.g., Echinacea, ginseng, peppermint, basil, rosemary, ginger, maca, or tisanes), a stimulant (e.g., guarana), an amino acid (e.g., L-theanine, phenylalanine, tyrosine, or tryptophan), a cannabinoid, and/or a nutraceutical, or medicinal ingredient (e.g., a vitamin, such as a vitamin D, vitamin C and/or vitamin A).

In some preferred embodiments, the combination of active ingredients further comprises vitamin C (ascorbic acid). Vitamin C acts as an antioxidant, helping to protect cells from the damage caused by free radicals. It is also used by the body to make collagen, a protein that may help wounds heal. In addition, vitamin C improves the absorption of iron from plant-based foods and helps the immune system work properly to protect the body from disease.

The inclusion of vitamin C may reduce tiredness and fatigue, and improve energy metabolism.

Where present, the vitamin C may be present in any suitable amount, such as in an amount of at least about 0.001% by weight, at least about 0.01% by weight, or at least about 0.1% by weight of the oral product. In some embodiments, the vitamin C may be present in an amount of no greater than about 20% by weight, no greater than about 10% by weight, or no greater than about 5% by weight of the oral product.

Where present, the vitamin C may be present in an amount of from about 0.01% to about 20% by weight, or from about 0.05% to about 10% by weight of the oral product. The vitamin C may preferably be present in an amount of from about 0.01% to about 5% by weight of the oral product.

In some embodiments, the combination of active ingredients further comprises ginseng. Ginseng is the root of plants in the genus Panax, which are characterised by the presence of unique steroid saponin phytochemicals (ginsenosides) and gintonin. The putative major active components of ginseng comprise more than 100 species specific triterpene saponins or ‘ginsenosides’. Ginseng and ginseng extracts also contain a range of other potentially bioactive components, including alkaloids, phytosterols, sesquiterpenes, and polyphenols. Ginseng finds use as a dietary supplement in energy drinks or herbal teas, and in traditional medicine.

The ginseng may include any suitable form of ginseng, such as Panax ginseng (Korean ginseng), Panax notoginseng (China ginseng) and Panax quinquefolius (American ginseng). The ginseng may also comprise Ashwagandha (Withania somnifera), commonly known as Indian Ginseng. The ginseng may be present in the form of ginseng extract, chopped ginseng, shredded ginseng or powdered ginseng. Preferably, the ginseng is included in the form of ginseng extract or powdered ginseng extract. The ginseng may white ginseng, fresh ginseng or red ginseng. In some embodiments, the ginseng is red ginseng, such as red ginseng extract or powdered red ginseng extract. It has been found that the addition of ginseng may boost brain function and/or increase cognitive effects. The inclusion of ginseng has been found to be beneficial in improving the cognitive effects of the composition, and reducing mental and physical fatigue in the user.

Where present, the ginseng may be included in any suitable amount, such as in an amount of at least about 0.0001% by weight, at least about 0.001% by weight or at least about 0.01% by weight of the oral product. The ginseng may be present in an amount of no more than about 10% by weight, such as no more than about 5% by weight of the oral product. In some embodiments, ginseng is present in an amount of from about 0.0001% to about 10% by weight of the oral product. In some embodiments, ginseng is present in an amount of from about 0.001% to about 5% by weight of the oral product. In some embodiments, the ginseng is present in an amount of from about 0.001% to about 3% by weight of the oral product. In some embodiments, the ginseng is present in an amount of from about 0.01% to about 2% by weight of the oral product.

In some preferred embodiments, the combination of active ingredients further comprises ginseng in an amount of from about 0.001% to about 1% by weight of the oral product, and preferably from about 0.01% to about 1% by weight of the oral product.

Where present, ginseng may in some embodiments (e.g. where the oral product is a liquid oral dosage form) be present in an amount of from about 0.01% to about 1.5% by weight of the oral product. In some embodiments (e.g. where the oral product is a liquid oral dosage form), the ginseng is present in an amount of from about 0.02% to about 1% by weight, such as from about 0.05% to about 0.75% by weight, preferably from about 0.1% to about 0.5% by weight of the oral product.

In some preferred embodiments, the combination of active ingredients comprises caffeine, a combination of B vitamins, taurine and ginseng.

In some preferred embodiments, the combination of active ingredients comprises caffeine, a combination of B vitamins, taurine and vitamin C.

In some preferred embodiments, the combination of active ingredients comprises caffeine, a combination of B vitamins, taurine, vitamin C, and ginseng.

In some embodiments, the combination of active ingredients comprises (i) from about 0.01% to about 10% caffeine by weight of the oral product, (ii) a combination of B vitamins, where the total amount of B vitamins is from about 0.001% to about 5% by weight of the oral product, and (iii) from about 0.001% to about 10% taurine by weight of the oral product. In some embodiments, the combination of active ingredients comprises (i) from about 0.1% to about 5% caffeine by weight of the oral product, (ii) a combination of B vitamins, where the total amount of B vitamins is from about 0.001% to about 1% by weight of the oral product, and (iii) from about 0.01% to about 5% taurine by weight of the oral product. In some embodiments, the combination of active ingredients comprises (i) from about 0.1% to about 1% caffeine by weight of the oral product, (ii) a combination of B vitamins, where the total amount of B vitamins is from about 0.01% to about 1% by weight of the oral product, and (iii) from about 0.1% to about 1% taurine by weight of the oral product. In each of these embodiments, the B vitamins are as defined hereinabove.

In some embodiments, the combination of active ingredients comprises (i) from about 0.1% to about 1% caffeine by weight of the oral product, (ii) a combination of B vitamins, where the total amount of B vitamins is from about 0.01% to about 1% by weight of the oral product, and (iii) from about 0.01% to about 0.5% taurine by weight of the oral product. The B vitamins are defined hereinabove.

In any of these embodiments, the combination of active ingredients may further comprise vitamin C in an amount of from about 0.1% to about 5% by weight of the oral product. In any of these embodiments, the combination of active ingredients may further comprise ginseng in an amount of from about 0.001% to about 5% by weight of the oral product, or from about 0.01% to about 1% by weight of the oral product. In any of these embodiments, the combination of active ingredients may further comprise vitamin C in an amount of from about 0.1% to about 5% by weight and ginseng in an amount of from about 0.01% to about 1% by weight of the oral product.

In some embodiments, the combination of active ingredients comprises (i) from about 0.01% to about 10% caffeine by weight of the oral product, (ii) a combination of B vitamins, where the total amount of B vitamins is from about 0.001% to about 5% by weight of the oral product, (iii) from about 0.001% to about 10% taurine by weight of the oral product, and (iv) from about 0.05% to about 10% vitamin C by weight of the oral product. In some embodiments, the combination of active ingredients comprises (i) from about 0.1% to about 5% caffeine by weight of the oral product, (ii) a combination of B vitamins, where the total amount of B vitamins is from about 0.001% to about 1% by weight of the oral product, (iii) from about 0.01% to about 5% taurine by weight of the oral product, and (iv) from about 0.1% to about 5% vitamin C by weight of the oral product. In some embodiments, the combination of active ingredients comprises (i) from about 0.1% to about 1% caffeine by weight of the oral product, (ii) a combination of B vitamins, where the total amount of B vitamins is from about 0.01% to about 1% by weight of the oral product, (iii) from about 0.01% to about 1% taurine by weight of the oral product, and (iv) (iii) from about 0.1% to about 1% vitamin C by weight of the oral product. In each of these embodiments, the B vitamins are as defined hereinabove.

In some embodiments, the combination of active ingredients comprises (i) from about 0.01% to about 5% caffeine by weight of the oral product, (ii) a combination of B vitamins, where the total amount of B vitamins is from about 0.001% to about 1% by weight of the oral product, (iii) from about 0.01% to about 5% taurine by weight of the oral product, and (iv) from about 0.1% to about 5% vitamin C by weight of the oral product. In each of these embodiments, the B vitamins are as defined hereinabove.

In some embodiments, the combination of active ingredients comprises (i) from about 0.1% to about 5% caffeine by weight of the oral product, (ii) a combination of B vitamins, where the total amount of B vitamins is from about 0.001% to about 1% by weight of the oral product, (iii) from about 0.01% to about 5% taurine by weight of the oral product, and (v) from about 0.01% to about 1% ginseng by weight of the oral product. In some embodiments, the combination of active ingredients comprises (i) from about 0.1% to about 1% caffeine by weight of the oral product, (ii) a combination of B vitamins, where the total amount of B vitamins is from about 0.01% to about 1% by weight of the oral product, (iii) from about 0.01% to about 1% taurine by weight of the oral product, and (v) from about 0.1% to about 1% ginseng by weight of the oral product. In each of these embodiments, the B vitamins are as defined hereinabove.

In some embodiments, the combination of active ingredients comprises (i) from about 0.1% to about 5% caffeine by weight of the oral product, (ii) a combination of B vitamins, where the total amount of B vitamins is from about 0.001% to about 1% by weight of the oral product, (iii) from about 0.01% to about 5% taurine by weight of the oral product, (iv) from about 0.1% to about 5% vitamin C by weight of the oral product, and (v) from about 0.01% to about 1% ginseng by weight of the oral product. In each of these embodiments, the B vitamins are as defined hereinabove.

The amounts of the active ingredients as described above have been found by the inventors to provide a beneficial effect of increasing energy levels and boosting brain function, while providing a safe product with reduced side effects. The amounts may be tailored such that the product has high efficacy while also ensuring consumer safety and avoiding any overdose of actives.

Other suitable additional active ingredients are described below.

L-theanine is an amino acid analogue that is also referred to as L-γ-glutamylethylamide and N⁵-ethyl-L-glutamine. Where present, L-theanine may be included in an amount of from about 0.01% to about 10% by weight of the oral product. The L-theanine may preferably be present in an amount of from about 0.1% to about 5% by weight of the oral product.

In some embodiments, the combination of active ingredients further comprises a botanical active ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). The botanical materials useful in the present disclosure may comprise, without limitation, any of the compounds and sources set forth herein, including mixtures thereof Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Passionflower, Guyasa, maca, matcha tea, beetroot juice, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, echinacea and Yerba mate.

In some embodiments, the combination of active ingredients further comprises Echinacea, which is a genus of herbaceous flowering plants in the daisy family, commonly referred to as coneflowers. Where present, Echinacea may be present in an amount of from about 0.001% to about 3% by weight of the oral product, and preferably from about 0.01% to about 1% by weight of the oral product.

The combination of active ingredients may further comprise a vitamin selected from vitamin A, vitamin D, vitamin E and vitamin K, or mixtures thereof. In some preferred embodiments, the combination of active ingredients further comprises vitamin D, optionally in an amount of from about 0.001% to about 1% by weight of the oral product.

The combination of active ingredients may further comprise an amino acid, such as an amino acid selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, gamma-aminobutyric acid (GABA), L-theanine, hydroxyproline, and beta-alanine. In some embodiments, the combination of active ingredients comprises L-tyrosine.

In some embodiments, the combination of active ingredients comprises one or more of choline, alpha GPC, theobromine, Guyasa, maca, matcha tea, methylliberine, nitrate or beetroot juice, yerba mate, glucose, or theacrine.

In some embodiments, the combination of active ingredients comprises a cannabinoid. The cannabinoid may be a derivative or extract of cannabis. Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids). Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids. In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof. In some embodiments, the cannabinoid is or comprises at least tetrahydrocannabinol (THC). In some embodiments, the cannabinoid is or comprises at least cannabidiol (CBD).

In some embodiments, the combination of active ingredients comprises a cannabinoid (such as cannabidiol) in an amount of at least about 0.001% by weight of the oral product, such as in a range from about 0.001% to about 10% by weight, such as from about 0.01% to about 5% by weight, such as from about 0.1% to about 2.5% by weight, such as from 0.5% to about 1% by weight of the oral product.

In some embodiments, the combination of active ingredients comprises magnesium glycinate. Where present, magnesium glycinate may be included in an amount of from about 0.1% to about 10% by weight, such as from about 0.5% to about 5% by weight, or from about 0.5% to about 1% by weight of the oral product.

For the avoidance of doubt, combinations of the above end points are explicitly envisaged by the present disclosure. This applies to any of the ranges disclosed herein.

### Ratios of Active Ingredients

The ratios of the active ingredients in the oral product may be selected to increase the cognitive effects of the oral product on the consumer.

In some embodiments, the caffeine and taurine and present in a weight ratio of from about 15:1 to about 1:15, such as from about 10:1 to about 1:10, preferably from about 5:1 to about 1:5 and more preferably from about 5:1 to about 1:1. In preferred embodiments, the caffeine and taurine may be present in a weight ratio of about 1:1. Alternatively, the caffeine and taurine may be present in a weight ratio of about 4:1.

In some embodiments, the caffeine and total amount of vitamin B are present in a weight ratio of from about 1:1 to about 15:1, such as from about 1:1 to about 10:1. In preferred embodiments, the caffeine and total amount of vitamin B are present in a weight ratio of from about 2:1 to about 10:1, or from about 3:1 to about 5:1.

In other embodiments, the caffeine and total amount of vitamin B are present in a weight ratio of from about 1:1 to about 30:1, preferably from about 10:1 to about 30:1. In preferred embodiments, the caffeine and total amount of vitamin B are present in a weight ratio of from about 15:1 to about 30:1.

In some embodiments, the taurine and total amount of vitamin B are present in a weight ratio of from about 10:1 to about 1:2, such as from about 7.5:1 to about 1:2. In some preferred embodiments, the taurine and total amount of vitamin B are present in a weight ratio of from about 7.5:1 to about 2:1. In other preferred embodiments, the taurine and total amount of vitamin B are present in a weight ratio of from about 1:1 to about 1:2.

Where present, the ratio of ginseng in the product may also provide advantages for improving cognition and mental focus. Where present, the ginseng may be present in an amount such that the weight ratio of caffeine to ginseng is from about 5:1 to about 1:5, such as from about 2:1 to about 1:2. Preferably, the ginseng may be present in an amount such that the weight ratio of caffeine to ginseng is from about 2:1 to about 1:1.

Where present, the ginseng may be present in an amount such that the weight ratio of taurine to ginseng is from about 5:1 to about 1:5. In some embodiments, the weight ratio of taurine to ginseng is from about 5:1 to about 1:1, such as from about 2:1 to about 1:1. In some embodiments, the weight ratio of taurine to ginseng is from about 1:1 to about 1:4.

Where present, the vitamin C may be included such that the weight ratio of vitamin C to caffeine is from about 5:1 to about 1:5. In some preferred embodiments, the weight ratio of vitamin C to or caffeine is from about 3:1 to about 1:3, more preferably from about 2:1 to about 1:1. Where present, the vitamin C may be included such that the weight ratio of vitamin C to taurine is from about 15:1 to about 1:5. In some preferred embodiments, the weight ratio of vitamin C to taurine is from about 10:1 to about 1:1, more preferably from about 10:1 to about 2:1.

In some embodiments, the oral product comprises a combination of active ingredients comprising:
(i) caffeine, (ii) a combination of B vitamins, and (iii) taurine,
wherein the weight ratio of caffeine to taurine is from about 5:1 to about 1:1;
wherein the weight ratio of caffeine to total amount of vitamin B is from about 1:1 to about 10:1; and
wherein the weight ratio of taurine to total amount of vitamin B is from about 10:1 to about 1:2.

In some embodiments, the oral product comprises a combination of active ingredients comprising:
(i) caffeine, (ii) a combination of B vitamins, and (iii) taurine,
wherein the weight ratio of caffeine to taurine is about 1:1, wherein the combination of B vitamins is as described in any of the embodiments hereinabove.

In some embodiments, the oral product is a liquid oral dosage form comprising a combination of active ingredients comprising:
(i) caffeine, (ii) a combination of B vitamins, and (iii) taurine,
wherein the weight ratio of caffeine to taurine is about 1:1, wherein the combination of B vitamins is as described in any of the embodiments hereinabove.

In some embodiments, the oral product comprises a combination of active ingredients comprising:
(i) caffeine, (ii) a combination of B vitamins, (iii) taurine, and (iv) ginseng,
wherein the weight ratio of caffeine to ginseng is from about 2:1 to about 1:1, and optionally
wherein the weight ratio of caffeine to taurine is from about 4:1 to about 1:1.

In some embodiments, the oral product comprises a combination of active ingredients comprising:
(i) caffeine, (ii) a combination of B vitamins, (iii) taurine, and (iv) ginseng, wherein the weight ratio of caffeine to taurine is from about 4:1 to about 1:1;
wherein the weight ratio of caffeine to total amount of vitamin B is from about 1:1 to about 10:1;
wherein the weight ratio of caffeine to ginseng is from about 2:1 to about 1:1.

### Additives

Depending on the type of oral product being processed, the product may include one or more additional components in addition to the combination of active ingredients. For example, the oral product may further comprise an additive selected from the group consisting of a flavouring agent, sweetener, buffering agent, acidifying agent, thickener, filler, binder, humectant, preservative, salt, colouring agent, oral care additive, disintegration aid, antioxidant, water or mixtures thereof. In some embodiments, the oral product further comprises one or more additives selected from the group consisting of a flavouring agent, sweetener, acidifying agent, thickener, filler, binder, humectant, preservative, and mixtures thereof.

In some embodiments, the oral product is preservative-free, that is to say there are no preservatives present in the oral product.

### Filler

Depending on the form of the product, the oral product may comprise a filler. Fillers, for example, may fulfil multiple functions, such as enhancing certain organoleptic properties such as texture and mouthfeel, enhancing cohesiveness or compressibility of the product, and the like.

In some embodiments, the filler is a porous particulate material and is cellulose-based. For example, the filler or bulking agent may be a non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. In some embodiments, the filler is a cellulose material selected from the group consisting of maize fiber, oat fiber, barley fiber, rye fiber, buckwheat fiber, sugar beet fiber, bran fiber, bamboo fiber, wood pulp fiber, cotton fiber, citrus pulp fiber, grass fiber, willow fiber, poplar fiber, cocoa fiber, derivatives thereof, and combinations thereof. In some embodiments, the filler is a cellulose material selected from the group consisting of sugar beet fiber, wood pulp fiber, bamboo fiber, derivatives thereof, and combinations thereof.

In some embodiments, the filler is derived from wood pulp fiber. One particularly suitable filler for use in the products described herein is microcrystalline cellulose (“MCC”). The MCC may be synthetic or semi-synthetic, or it may be obtained entirely from natural celluloses. The MCC may be selected from the group consisting of AVICEL® grades PH-100, PH-101, PH-102, PH-103, PH-105, PH-112, PH-113, PH-200, PH-300, PH-301, PH-302, VIVACEL® grades 101, 102, 12, 20 and EMOCEL® grades 50M and 90M, and the like, and mixtures thereof.

In some embodiments, the filler is a non-tobacco plant material or a derivative thereof. Non-limiting examples of derivatives of non-tobacco plant material include starches (e.g., from potato, wheat, rice, corn), natural cellulose, and modified cellulosic materials. Additional examples of potential fillers include maltodextrin, dextrose, calcium carbonate, calcium phosphate, lactose, mannitol, xylitol, and sorbitol. Combinations of these fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the product based on the ability of the starch material to impart a specific organoleptic property to product. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “modified” starches. Other starches are obtained and subsequently modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, enzyme treatment, acetylation, hydroxypropylation, and/or partial hydrolysis. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

### Water

The moisture content (e.g., water content) of the oral product, prior to use by a consumer of the product, may vary according to the desired properties.

Typically, for a solid oral product, prior to insertion into the mouth of the user, the water content is less than about 60% by weight, and generally is from about 1 to about 60% by weight, for example, from about 5 to about 55% by weight, about 10 to about 50% by weight, about 20 to about 45% by weight, or about 25 to about 40% by weight of the oral product; including water amounts of at least about 5% by weight, at least about 10% by weight, at least about 15% by weight, and at least about 20% by weight of the oral product.

For a liquid oral product, the water content may be higher and may be at least about 50%, such as at least about 60%, such as at least about 75% by weight of the oral product. Preferably, the water content of a liquid oral product may be at least about 90% by weight of the oral product. Preferably, the water content of a liquid oral product may be from about 60% to about 99.9% by weight, such as from about 75% to about 99% by weight, such as from about 80% to about 98% by weight of the oral product.

### Flavouring Agent

As used herein, the term “flavouring agent” (or “flavour” or “flavourant”) refers to materials which, where local regulations permit, may be used to create a desired taste, aroma or other somatosensorial sensation in a product for adult consumers. Examples of sensory characteristics that can be modified by the flavoring agent include taste, mouthfeel, moistness, coolness/heat, and/or fragrance/aroma. Flavouring agents may be natural or synthetic, and the character of the flavours imparted thereby may be described, without limitation, as fresh, sweet, herbal, confectionary, floral, fruity, or spicy.

The flavouring agent may be selected from the group consisting of naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, raspberry, strawberry, peach, apple, orange, mango, pineapple, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, coconut, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., honey, sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavouring agent comprises a natural flavoring, such as berry (e.g. raspberry, blueberry or strawberry), honey, citrus (such as lemon, bergamot, orange or lime), or other botanical material.

In some embodiments, the flavouring agent comprises menthol, spearmint and/or peppermint. In some embodiments, the flavouring agent comprises flavour components of cucumber, blueberry, citrus fruits and/or redberry. In some embodiments, the flavouring agent comprises eugenol. In some embodiments, the flavouring agent comprises flavour components extracted from tobacco. In some embodiments, the flavouring agent comprises flavour components extracted from cannabis.

In some embodiments, the flavouring agent may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

Where present, a flavouring agent may be included in the oral product in an amount up to about 10% by weight, such as up to about 5% by weight, such as up to about 1% by weight of the oral product. In some embodiments, a flavouring agent is present in an amount of from about 0.01% to about 5% by weight, preferably in an amount of from about 0.1% to about 2.5% by weight of the oral product, and more preferably in an amount of from about 0.25% to about 1% by weight of the oral product.

### Binding Agent

In some embodiments, the oral product may further comprise at least one binder. A binder (or combination of binders) may be employed in the product in certain embodiments, in amounts sufficient to provide the desired physical attributes and physical integrity to the product. Binders can be organic or inorganic, or a combination thereof. Representative binders include cellulose derivatives, povidone, sodium alginate, starch-based binders, pectin, carrageenan, pullulan, zein, and the like, and combinations thereof. The amount of binder utilised in the product can vary, but may be up to about 30% by weight, and certain embodiments are characterised by a binder content of at least about 0.1% by weight, such as from about 1% to about 30% by weight, or about 1% to about 10% by weight, based on the total weight of the oral product.

In some embodiments, the binder comprises a cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In some embodiments, the cellulose derivative is or comprises HPC. In some embodiments, the cellulose derivative is a combination of HPC and HPMC. In some embodiments, the oral product comprises from about 1% to about 10% of the cellulose derivative (such as HPC) by weight of the oral product, with certain embodiments comprising from about 1% to about 5% by weight of cellulose derivative (such as HPC), based on the weight of the product.

### Humectants

In some embodiments, the oral product comprises at least one humectant. Examples of suitable humectants that may be included in the product include, but are not limited to, glycerin, 1,2-propanediol (propylene glycol), 1,3-propanediol, dipropylene glycol, sorbitol, xylitol, mannitol, and the like. In some embodiments, the humectant is or comprises glycerin. In some embodiments, the oral product comprises glycerin. In some embodiments, the humectant is or comprises propylene glycol. In some embodiments, the oral product comprises propylene glycol. The amount of humectant utilised in the oral product can vary, but may be up to about 5% by weight, and certain embodiments can be characterised by a humectant content of at least about 1% by weight, such as about 2 to about 5% by weight of the oral product. In some embodiments, the humectant (such as glycerin and/or propylene glycol) may be present in an amount of from about 0.01% to about 25% by weight of the oral product, such as from about 0.1% to about 20% by weight of the oral product, such as from about 0.5% to about 15% by weight of the oral product, such as from about 1% to about 10% by weight of the oral product, such as from about 5% to about 10% by weight of the oral product.

### Sweeteners

In order to improve the sensory properties of the oral product, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame and the like.

In some embodiments, the sweetener comprises one or more sugar alcohols. The sugar alcohol may include erythritol, arabitol, ribitol, isomalt, maltitol, dulcitol, iditol, mannitol, xylitol, lactitol, sorbitol, or combinations thereof. In some embodiments, the sweetener is selected from the group consisting of fructose, sucrose, glucose, maltose, mannose, galactose, lactose, stevia, honey, sucralose, isomaltulose, maltodextrin, saccharin, aspartame, acesulfame K, neotame, erythritol, arabitol, ribitol, isomalt, maltitol, dulcitol, iditol, mannitol, xylitol, lactitol, sorbitol, and mixtures thereof.

In some embodiments, the sweetener is selected from the group consisting of sucralose, acesulfame K, aspartame, maltodextrin, mannitol, sucrose, and mixtures thereof. Preferably, the sweetener may be sucralose and/or acesulfame K. When present in the oral product, the sweetener (such as sucralose and/or acesulfame K) may be present in an amount of from about 0.001% to about 5% by weight, such as from about 0.01% to about 3% by weight, preferably from about 0.01% to about 1% by weight of the oral product.

### Buffering Agents

Non-limiting examples of suitable buffering agents that may be included in the oral product include alkali metals acetates, glycinates, phosphates, glycerophosphates, citrates, carbonates, hydrogen carbonates, borates, or mixtures thereof. In some embodiments in which a buffering agent is present, the buffering agent is selected from the group consisting of sodium carbonate, sodium bicarbonate, sodium phosphate, ammonium phosphate, dicalcium phosphate, tricalcium phosphate, and mixtures thereof. In some embodiments, the buffering agent is sodium bicarbonate and/or sodium carbonate. Where present, the buffering agent (e.g. sodium bicarbonate and/or sodium carbonate) may be included in an amount less than about 5% based on the weight of the oral product; for example, from about 0.5% to about 5%, such as, e.g., from about 0.75% to about 4%, from about 0.75% to about 3%, or from about 1% to about 2% by weight, based on the total weight of the oral product.

### Organic Acid

In some embodiments, the product comprises an organic acid. As used herein, the term “organic acid” refers to an organic (i.e., carbon-based) compound that is characterised by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific mixture ingredient as opposed to merely being inherently present as a component of another mixture ingredient (e.g., the small amount of organic acid which may inherently be present in a mixture ingredient such as a tobacco material). In some embodiments, the one or more organic acids are added neat (i.e., in their free acid, native solid or liquid form) or as a solution in, e.g. water. In some embodiments, the one or more organic acids are added in the form of a salt.

In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

In some embodiments, the organic acid is an alkyl carboxylic acid. Non-limiting examples of alkyl carboxylic acids include formic acid, acetic acid, propionic acid, octanoic acid, nonanoic acid, decanoic acid, undecanoic acid, dodecanoic acid, stearic acid, oleic acid, linoleic acid, linolenic acid, and the like. In some embodiments, the organic acid is an alkyl sulfonic acid. Non-limiting examples of alkyl sulfonic acids include propanesulfonic acid and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like. In some preferred embodiments, the organic acid is citric acid, sodium citrate, calcium citrate, or a combination thereof.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Additional non-limiting examples of suitable organic acids include 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), camphoric acid (+), camphor-10-sulfonic acid (+), capric acid, caproic acid, caprylic acid, cinnamic acid, cyclamic acid, decanoic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glycerophosphoric acid, glycolic acid, hippuric acid, isobutyric acid, lactobionic acid, lauric acid, malonic acid, mandelic acid, methanesulfonic acid, naphthalene-1,5-disulfonic acid, naphthalene-2-sulfonic acid, oleic acid, palmitic acid, pamoic acid, pyroglutamic acid, sebacic acid, stearic acid, and undecylenic acid.

Preferably, the organic acid is selected from the group consisting of citric acid, malic acid, lactic acid, benzoic acid, tartaric acid, and mixtures thereof. In some preferred embodiments, the organic acid is or comprises citric acid or a salt thereof.

In some embodiments, the organic acid is or comprises citric acid anhydrate.

The amount of organic acid present in the product may vary. The oral product may comprise from about 0.01% to about 10% by weight of organic acid (e.g. citric acid or a salt thereof), present as one or more organic acids, based on the total weight of the oral product. In some embodiments, the oral product comprises at least about 0.01%, at least about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or at least about 10% organic acid by weight, based on the total weight of the oral product. In some preferred embodiments, the oral product comprises from about 0.01% to about 5% by weight of organic acid (e.g. citric acid or salt thereof) based on the weight of the oral product. For example, the oral product comprises an organic acid in an amount of from about 0.1% to about 2.5% by weight of the oral product. In the case where a salt of an organic acid is added (e.g., citric acid anhydrate), the percent by weight is calculated based on the weight of the free acid, not including any counter-ion which may be present.

In certain embodiments the organic acid inclusion is sufficient to provide a product pH of from about 4.0 to about 9.0, such as from about 4.5 to about 7.0, or from about 5.5 to about 7.0, from about 4.0 to about 5.5, or from about 7.0 to about 9.0. In some embodiments, the organic acid inclusion is sufficient to provide a product pH of from about 4.5 to about 6.5, for example, from about 4.5, about 5.0, or about 5.5, to about 6.0, or about 6.5. In some embodiments, the organic acid is provided in a quantity sufficient to provide a pH of the product of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In other embodiments, a mineral acid (e.g., hydrochloric acid, sulfuric acid, phosphoric acid, or the like) is added to adjust the pH of the product to the desired value.

Organic acids (e.g., citric acid) may be added neat (i.e., as a solid) or in solution, for example, in water. In some embodiments, the organic acid is added as a 50% aqueous solution.

### Salts

The oral product may further comprise a salt. This may be included in an amount sufficient to provide desired sensory attributes to the product. Non-limiting examples of suitable salts include sodium chloride, potassium chloride, ammonium chloride, flour salt, sodium acetate, sodium citrate, and the like. The salt may be included in any suitable amount, such as at least about 0.5% by weight, such as at least about 1% by weight, such as at least about 1.5% by weight of the oral product. In some embodiments, the oral product may comprise salt in an amount of from about 0.5% to about 10% by weight, such as from about 1% to about 7.5% by weight, such as from about 1.5% to about 5% by weight, based on the total weight of the oral product.

### Thickeners

The oral product may comprise a thickening agent in some embodiments. Suitable thickening agents may include a hydrocolloid, such as xanthan gum, guar gum, konjac gum, gum tragacanth and gum Arabic. For example, xanthan gum is understood to be a thickening agent that thickens compositions when added during cold processing (i.e. when heat is not applied). Where present, a thickening agent (e.g. xanthan gum) may be included in an amount of from about 0.001% to about 5% by weight, and preferably from about 0.01% to about 1% by weight of the oral product.

### Colouring Agents

A colouring agent may be employed in amounts sufficient to provide the desired physical attributes to the product. Examples of colouring agents include various dyes and pigments, such as caramel colouring and titanium dioxide. Natural colouring agents such as curcumin, beet juice extract, spirulina; also a variety of synthetic pigments may also be used. The amount of colorant utilised in the oral product can vary, but when present is typically up to about 3% by weight, such as from about 0.1%, about 0.5%, or about 1%, to about 3% by weight, based on the total weight of the oral product.

### Other Additives

Other ingredients such as preservatives (e.g., potassium sorbate), disintegration aids (e.g., croscarmellose sodium, crospovidone, sodium starch glycolate, pregelatinized corn starch, and the like), and/or antioxidants can also be used. Typically, such ingredients, where used, are used in amounts of up to about 10% by weight, for example at least about 0.1% by weight, such as about 0.5 to about 10% by weight of the oral product. A disintegration aid may be employed in an amount sufficient to provide control of desired physical attributes of the oral product such as, for example, by providing loss of physical integrity and dispersion of the various component materials upon contact of the formulation with water (e.g., by undergoing swelling upon contact with water).

Examples of further types of additives include zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No 2007/0062549 to Holton, Jr. et al., each of which is incorporated herein by reference. Typical inclusion ranges for such additional additives can vary depending on the nature and function of the additive and the intended effect on the final product, with an example range of up to about 10% by weight, based on total weight of the oral product (e.g., about 0.1 to about 5% by weight).

In some embodiments, the oral product comprises a magnesium salt. A non-limiting example of a suitable magnesium salt is magnesium gluconate. In some embodiments, the oral product comprises magnesium in an amount by weight from about 0.1% to about 2%, or from about 0.2 to about 1%, based on elemental magnesium.

In some preferred embodiments, the oral product comprises zinc or zinc gluconate. In some embodiments, the oral product comprises zinc or zinc gluconate in an amount by weight from about 0.1% to about 2%, or from about 0.1 to about 1%, based on the oral product.

The aforementioned additives can be employed together (e.g., as additive formulations) or separately (e.g., individual additive components can be added at different stages involved in the preparation of the final product). Furthermore, the aforementioned types of additives may be encapsulated as provided in the final product or composition. Exemplary encapsulated additives are described, for example, in WO 2010/132444 to Atchley, which has been previously incorporated by reference herein.

### Oral Product

The products or compositions as described herein are configured for oral use. The term “configured for oral use” as used herein means that the product is provided in a form such that during use, saliva in the mouth of the user causes one or more of the components of the product (e.g., active ingredients) to pass into the mouth of the user. In certain embodiments, the product is adapted to deliver active ingredients and optionally flavouring agent to a user through mucous membranes in the user's mouth, the user's digestive system, or both. In some instances, the active ingredients and optionally flavouring agent can be absorbed through the mucous membranes in the mouth or absorbed through the digestive tract when the product is used.

In some embodiments, the oral product comprises the combination of active ingredients in an amount of at least about 0.01% by weight, such as at least about 0.1% by weight, or preferably at least about 1% by weight of the oral product. The oral product may, for example, comprise the combination of active ingredients in an amount of at least about 0.25% by weight, at least about 0.3% by weight, at least about 0.5% by weight, at least about 0.75% by weight, at least about 1% by weight, at least about 1.5% by weight, at least about 2% by weight, at least about 3% by weight, at least about 4% by weight, or at least about 5% by weight of the oral product. The combination of active ingredients may be present in an amount of no greater than about 50% by weight, such as no greater than about 40%, such as no greater than about 30%, such as no greater than about 20% by weight, such as no greater than about 10% of the oral product.

The combination of active ingredients may be present in an amount of from about 0.01% to about 20% by weight. For example, the combination of active ingredients may be present in an amount of from about 0.05% to about 15% by weight, such as from about 0.1% to about 10% by weight, or from about 0.5% to about 5% by weight of the oral product. Preferably, the combination of active ingredients is present in an amount of from about 0.1% to about 10% by weight of the oral product. Preferably, the combination of active ingredients is present in an amount of from about 0.5% to about 5% by weight of the oral product, more preferably from about 1% to about 5% by weight of the oral product.

The oral product may take any form that is suitable for application to the oral cavity of a human or animal. In some embodiments, the oral product is an oral dosage form in the form of a solid, a gel or a liquid. In some embodiments, the oral product is a solid oral dosage form.

In some preferred embodiments, the oral product is a liquid oral dosage form.

An oral product as described herein may take various forms, including gels, melts, tablets, lozenges, powders, pouches, and liquids (e.g. beverages).

### Liquid Oral Dosage Forms

In some preferred embodiments, the oral product is in the form of a liquid dosage form. The liquid dosage form is suitable for oral consumption, such that it may be referred to as a beverage as it can be ingested (i.e. drunk) by the user.

The liquid oral dosage form may be in the form of a shot; i.e. a drink that may be consumed quickly, e.g. in a single gulp or a couple of gulps. Formulating the oral product in the form of a shot may be advantageous as it provides a convenient and easy-to-use mode of administration that does not take up much room during storage (e.g. in a fridge or cupboard) and is easily portable by the user so can be taken on the go. Shots may also provide an efficient delivery vehicle for actives, whereby a level of active sufficient to provide good efficacy can be delivered to the user without the need to consume large volumes of liquid. Additionally, the shots may be formulated to have a pleasant taste. Drinking a shot may be considered to be more palatable and easier to a user than swallowing a tablet, for example.

The liquid oral dosage form may also be in the form of a larger beverage that is consumed more slowly over several gulps. The liquid oral dosage form may have a volume of from about 1 mL to about 250 mL, such as from about 1 mL to about 200 mL. The liquid oral dosage form may have a volume of from about 100 mL to about 250 mL and be provided in a package such as a carton, cup, can or bottle of liquid.

The liquid oral dosage form may have a volume of from about 10 mL to about 100 mL, such as from about 25 mL to about 75 mL. In such embodiments, the liquid oral dosage form may be considered to be a shot. In some preferred embodiments, the liquid oral dosage form may have a volume of from about 50 mL to about 100 mL. For example, the volume of the liquid oral dosage form may be from about 50 mL to about 75 mL. The volume of liquid may be about 60 mL.

When in the form of a liquid oral dosage form, the oral product may further comprise water in an amount of from about 50% to about 99.9% by weight of the oral product. In some embodiments, the oral product comprises water in an amount of from about 75% to about 99.5% by weight, such as from about 80% to about 99% by weight, such as from about 90% to about 97.5% by weight of the oral product. In some embodiments, the oral product comprises water in an amount of from about 90% to about 99.5% by weight of the oral product, and may be from about 95% to about 99% by weight of the oral product.

Where present, the water may include tap water, rain water, mineralised water, or distilled water. Preferably, the water is distilled water.

The liquid oral dosage form may comprise the combination of active ingredients in an amount of from about 0.1% to about 10% by weight of the oral product, and water in an amount of from about 90% to about 99.9% by weight of the oral product. The liquid oral dosage form may comprise the combination of active ingredients in an amount of from about 0.5% to about 5% by weight of the oral product, and water in an amount of from about 90% to about 99.5% by weight of the oral product.

In some embodiments, the combination of active ingredients in the liquid oral dosage form comprises (i) caffeine; (ii) a combination of B vitamins as defined hereinabove; (iii) taurine; and optionally (iv) vitamin C and/or (v) ginseng. The combination of active ingredients in the liquid oral dosage form may comprise (i) caffeine; (ii) a combination of B vitamins as defined hereinabove; (iii) taurine; (iv) vitamin C; and (v) ginseng.

In any of the above embodiments, the liquid oral dosage form may further comprise zinc or zinc gluconate, optionally in an amount of from about 0.1% to about 2% by weight of the oral product.

In any of the above embodiments, the liquid oral dosage form may further comprise an organic acid, preferably citric acid or a salt thereof. The organic acid may be present in an amount of from about 0.01% to about 5% by weight of the oral product.

The liquid oral dosage form (e.g. in the form of a shot having a volume of 50 mL to 100 mL) may comprise caffeine in an amount of from about 50 mg to about 500 mg, preferably from about 100 mg to about 250 mg, more preferably from about 100 mg to about 200 mg. In some embodiments, the liquid oral dosage form (e.g. in the form of a shot having a volume of 50 ml to 100 mL) comprises caffeine in an amount of from about 100 mg to about 150 mg. The skilled person will understand that the “mg” value for caffeine refers to the active ingredient. A naturally derived caffeine may, for example, have a % caffeine and need to be included in an amount so as to deliver caffeine in the above-defined amounts. A 60% caffeine green tea extract, for instance, would need to be included at 250 mg to deliver 150 mg of caffeine.

The liquid oral dosage form (e.g. in the form of a shot having a volume of 50 mL to 100 mL) may comprise taurine in an amount of from about 1 mg to about 500 mg, preferably from about 10 mg to about 250 mg, more preferably from about 20 mg to about 200 mg. In some embodiments, the liquid oral dosage form (e.g. in the form of a shot having a volume of 50 mL to 100 mL) comprises taurine in an amount of from about 100 mg to about 200 mg. In some embodiments, the liquid oral dosage form (e.g. in the form of a shot having a volume of 50 mL to 100 mL) comprises taurine in an amount of from about 10 mg to about 50 mg.

The liquid oral dosage form (e.g. in the form of a shot having a volume of 50 mL to 100 mL) may comprise a total amount of B vitamins in an amount of from about 1 mg to about 100 mg, preferably from about 10 mg to about 75 mg, more preferably from about 25 mg to about 50 mg.

In some preferred embodiments, the liquid oral dosage form (e.g. in the form of a shot having a volume of 50 mL to 100 mL) comprises a combination of vitamins B1, B2, B3, B5, B6, B7, B9 and B12, where the total amount of B vitamins is from about 10 mg to about 50 mg. In other preferred embodiments, the liquid oral dosage form (e.g. in the form of a shot having a volume of 50 mL to 100 mL) comprises a combination of vitamins B1, B2, B6, and B7, where the total amount of B vitamins is from about 1 mg to about 25 mg.

Where present, the liquid oral dosage form (e.g. in the form of a shot having a volume of 50 mL to 100 mL) may comprise ginseng in an amount of from about 10 mg to about 300 mg, preferably from about 25 mg to about 200 mg, more preferably from about 50 mg to about 150 mg.

Where present, the liquid oral dosage form (e.g. in the form of a shot having a volume of 50 mL to 100 mL) may include vitamin C in an amount of from about 10 mg to about 500 mg, such as from about 50 mg to about 500 mg, such as from about 200 mg to about 400 mg. In some preferred embodiments, the liquid oral dosage form (e.g. in the form of a shot having a volume of 50 mL to 100 mL) may include vitamin C in an amount of from about 250 mg to about 350 mg.

In some embodiments, the liquid oral dosage form (e.g. in the form of a shot having a volume of 50 mL to 100 mL) comprises:
(i) from about 50 mg to about 500 mg of caffeine;
(ii) from about 1 mg to about 100 mg of a combination of B vitamins, wherein the combination of B vitamins comprises vitamin B2, vitamin B3, vitamin B6, vitamin B9 and vitamin B12; and
(iii) from about 1 mg to about 500 mg taurine;
optionally (iv) from about 50 mg to about 500 mg of vitamin C; and/or
optionally (v) from about 10 mg to about 300 mg of ginseng.

In some embodiments, the liquid oral dosage form (e.g. in the form of a shot having a volume of 50 mL to 100 mL) comprises:
(i) from about 50 mg to about 500 mg of caffeine;
(ii) from about 1 mg to about 100 mg of a combination of B vitamins, wherein the combination of B vitamins comprises vitamin B1, vitamin B2, vitamin B6, and vitamin B7; and
(iii) from about 1 mg to about 500 mg taurine;
optionally (iv) from about 10 mg to about 500 mg of vitamin C; and/or
optionally (v) from about 10 mg to about 300 mg of ginseng.

In some embodiments, the liquid oral dosage form (e.g. in the form of a shot having a volume of 50 mL to 100 mL) comprises:
(i) from about 100 mg to about 200 mg of caffeine;
(ii) from about 10 mg to about 50 mg of a combination of B vitamins, wherein the combination of B vitamins comprises vitamin B2, vitamin B3, vitamin B6, vitamin B9 and vitamin B12; and
(iii) from about 20 mg to about 200 mg taurine;
optionally (iv) from about 250 mg to about 350 mg of vitamin C; and/or
optionally (v) from about 50 mg to about 200 mg of ginseng.

In some embodiments, the liquid oral dosage form (e.g. in the form of a shot having a volume of 50 mL to 100 mL) comprises:
(i) from about 100 mg to about 200 mg of caffeine;
(ii) from about 1 mg to about 50 mg of a combination of B vitamins, wherein the combination of B vitamins comprises vitamin B1, vitamin B2, vitamin B6, and vitamin B7; and
(iii) from about 20 mg to about 200 mg taurine;
optionally (iv) from about 10 mg to about 350 mg of vitamin C; and/or
optionally (v) from about 50 mg to about 200 mg of ginseng.

In some embodiments, the liquid oral dosage form (e.g. in the form of a shot having a volume of 50 mL to 100 mL) comprises:
(i) from about 100 mg to about 200 mg of caffeine;
(ii) from about 10 mg to about 50 mg of a combination of B vitamins, wherein the combination of B vitamins comprises vitamin B2, vitamin B3, vitamin B6, vitamin B9 and vitamin B12;
(iii) from about 20 mg to about 200 mg taurine;
(iv) from about 250 mg to about 350 mg of vitamin C; and
(v) from about 50 mg to about 200 mg of ginseng.

In some embodiments, the liquid oral dosage form (e.g. in the form of a shot having a volume of 50 mL to 100 mL) comprises:
(i) from about 100 mg to about 200 mg of caffeine;
(ii) from about 1 mg to about 50 mg of a combination of B vitamins, wherein the combination of B vitamins comprises vitamin B1, vitamin B2, vitamin B6, and vitamin B7;
(iii) from about 20 mg to about 200 mg taurine;
(iv) from about 250 mg to about 350 mg of vitamin C; and
(v) from about 50 mg to about 200 mg of ginseng.

The amounts of the active ingredients as described above have been found by the inventors to provide a beneficial effect of increasing energy levels and boosting brain function, while providing a safe product with reduced side effects. The amounts may be tailored such that the product has high efficacy while also ensuring consumer safety and avoiding any overdose of actives.

The liquid oral dosage form may in some embodiments further comprise an acidifying agent. The acidifying agent may be an organic acid as described hereinabove. In some embodiments, the acidifying agent is or comprises citric acid or a salt thereof (e.g. citric acid anyhydrate). In some embodiments, the pH of the liquid oral dosage form is from about 2 to about 6, such as from about 2.5 to about 4 or from about 2.5 to about 3.5. In any of the above embodiments, the liquid oral dosage form may further comprise an organic acid, preferably citric acid or a salt thereof, in an amount of from about 100 mg to about 200 mg.

In any of the above embodiments, the liquid oral dosage form may further comprise zinc gluconate, optionally in an amount of from about 50 mg to about 150 mg.

The liquid oral dosage form may include any further suitable additive. Suitable additives are described in greater detail above, and all of the additives described herein may be included in the liquid oral dosage form. In some embodiments, the liquid oral dosage form may further comprise an additive selected from the group consisting of a flavouring agent, sweetener, acidifying agent, thickener, humectant, preservative, and mixtures thereof. Examples of each of these forms of additives are described hereinabove.

### Solid Oral Dosage Forms

In some embodiments, the oral product is in the form of a solid. As used herein, the term “solid” means that the products can substantially sustain their physical shape when unsupported by external means, e.g. packaging etc. Thus, they are considered to be solid, solid-like, in solid form or in solid-like form at room temperature. For the avoidance of doubt the solid product remains substantially solid at up to 30° C. In some embodiments, the oral product is in solid form, such as in the form of loose moist snuff, loose dry snuff, pelletized pieces, extruded or formed strips, pieces, rods, or sticks, finely divided ground powders, finely divided or milled agglomerates of powdered pieces and components, flake-like pieces, molded processed pieces, films, readily water-dissolvable or water-dispersible films or strips, capsule-like materials, tablets, lozenges. In some embodiments, the oral product is a tablet or lozenge. In some embodiments, the oral product is in the form of moist snuff or snus, which may or may not contain tobacco.

The oral products as disclosed herein can be formed into a variety of shapes, including pills, tablets, spheres, strips, films, sheets, coins, cubes, beads, ovoids, obloids, cylinders, bean-shaped, sticks, or rods. Cross-sectional shapes of the product can vary, and example cross-sectional shapes include circles, squares, ovals, rectangles, and the like. Such shapes can be formed in a variety of manners using equipment such as moving belts, nips, extruders, granulation devices, compaction devices, and the like.

In some embodiments, the solid oral product is in a form selected from the group consisting of a melt, a tablet or a lozenge.

### Melts

In some embodiments, the product can be meltable as discussed, for example, in US Patent App. Pub. No. 2012/0037175 to Cantrell et al., incorporated by reference herein in its entirety. As used herein, “melt,” “melting,” and “meltable” refer to the ability of the product to change from a solid state to a liquid state. That is, melting occurs when a substance (e.g., a product as disclosed herein) changes from solid to liquid, usually by the application of heat.

The application of heat in regard to a product as disclosed herein is provided by the internal temperature of a user's mouth. Thus, the term “meltable” refers to a product that is capable of liquefying in the mouth of the user as the product changes phase from solid to liquid, and is intended to distinguish products that merely disintegrate in the oral cavity through loss of cohesiveness within the product that merely dissolve in the oral cavity as aqueous-soluble components of the product interact with moisture.

Generally, meltable products comprise a lipid. In some embodiments, the composition comprises a lipid. The lipid is typically a fat, oil, or wax substance derived from animal or plant material (e.g., plant-derived fats), and typically comprises mostly triglycerides along with lesser amounts of free fatty acids and mono- or diglycerides. In certain embodiments, the lipid is a solid or semi-solid at room temperature (i.e., 25° C.) and capable of at least partially liquefying when subjected to the temperature of the oral cavity of the user (i.e., “melting”). Example plant-derived fats are comprised primarily of saturated or unsaturated fatty acid chains (most of which are bound within triglyceride structures) having a carbon length of about 10 to about 26 carbon atoms, or about 14 to about 20 carbon atoms, or about 14 to about 18 carbon atoms.

In some embodiments, the lipid comprises an oil and, in particular, a food grade oil, including fractionated oils. Such oils include, but are not limited to, vegetable oils (e.g., acai oil, almond oil, amaranth oil, apricot oil, apple seed oil, argan oil, avocado oil, babassu oil, beech nut oil, ben oil, bitter gourd oil, black seed oil, blackcurrant seed oil, borage seed oil, borneo tallow nut oil, bottle gourd oil, brazil nut oil, buffalo gourd oil, butternut squash seed oil, cape chestnut oil, canola oil, carob cashew oil, cocoa butter, cocklebur oil, coconut oil, corn oil, cothune oil, coriander seed oil, cottonseed oil, date seed oil, dika oil, egus seed oil, evening primrose oil, false flax oil, flaxseed oil, grape seed oil, grapefruit seed oil, hazelnut oil, hemp oil, kapok seed oil, kenaf seed oil, lallemantia oil, lemon oil, linseed oil, macadamia oil, mafura oil, marula oil, meadowfoam seed oil, mongongo nut oil, mustard oil, niger seed oil, nutmeg butter, okra seed oil, olive oil, orange oil, palm oil, papaya seed oil, peanut oil, pecan oil, perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, ramtil oil, rapeseed oil, rice bran oil, royle oil, sacha inchi oil, safflower oil, sapote oil, seje oil, sesame oil, shea butter, soybean oil, sunflower oil, taramira oil, tea seed oil, thistle oil, tigernut oil, tobacco seed oil, tomato seed oil, walnut oil, watermelon seed oil, wheat germ oil, and combinations thereof), animal oils (e.g., cattle fat, buffalo fat, sheep fat, goat fat, pig fat, lard, camel fat, tallow, liquid margarine, fish oil, fish liver oil, whale oil, seal oil, and combinations thereof), and mineral oils.

In certain embodiments, the plant-derived fats of the present disclosure include palm oil, (including fractionated palm oil) palm kernel oil, soybean oil, cottonseed oil, and mixtures thereof. In one embodiment, the lipid is a blend of palm oil and palm kernel oil. The lipid can be, for example, hydrogenated, partially hydrogenated, or non-hydrogenated. Example embodiments of lipids can be purchased under the brand names CEBES®, CISAO®, or CONF AO®, available from AarhusKarlshamn USA Inc.

The melting point of the lipid is typically about 29° C. or above, such as about 29° C. to about 49° C., or about 36° C. to about 45° C., or about 38° C. to about 41° C. In some embodiments, use of lipids with a melting point of less than about 36° C. is not advantageous due to possible melting during product storage or handling. One test for determining the melting point of lipids is the Mettler dropping point method (ASTM D3954-15, Standard Test Method for Dropping Point of Waxes, ASTM International, West Conshohocken, PA, 2015, www.astm.org).

When present, the amount of lipid within the composition may vary. In certain embodiments, the amount of lipid is at least about 10%, at least about 20%, or at least about 30%, on a dry weight basis of the composition. In certain embodiments, the amount of lipid is less than about 70%, less than about 60%, or less than about 50%, on a dry weight basis. Example lipid weight ranges include about 10 to about 70% dry weight, such as about 35 to about 50% dry weight. In some embodiments, the amount of lipid is about 35, about 40, about 45, or about 50% by weight of the total oral product.

In some embodiments, the oral product comprises a lipid. In one embodiment, the lipid is an oil selected from the group consisting of palm oil, palm kernel oil, soybean oil, sunflower oil, cottonseed oil, coconut oil, and combinations thereof, wherein the oil may be hydrogenated, partially hydrogenated, or non-hydrogenated. In one embodiment, the lipid is a trans-hydrogenated filling fat of medium hardness such as Confao® 5, available from AarhusKarlshamn USA Inc., 131 Marsh Street, Port Newark, NJ 07114.

In some embodiments, the product in meltable form comprises a lipid in an amount of from about 35 to about 50% by weight of the oral product, and a sugar alcohol in an amount of from about 35 to about 55% by weight of the oral product. In some embodiments, the sugar alcohol is isomalt, erythritol, sorbitol, arabitol, ribitol, maltitol, dulcitol, iditol, mannitol, xylitol, lactitol, or a combination thereof. In some embodiments, the sugar alcohol is or comprises isomalt. In some embodiments, a sugar substitute may be an alternative to the sugar alcohol, or used in combination with one or more sugar alcohols. Suitable sugar substitutes include allulose, soluble tapioca fiber, inulin, and combinations thereof.

### Tablets

In certain embodiments, the product is in the form of a compressed or molded tablet. An exemplary tablet dosage form weighs from about 250 mg to about 1500 mg, such as about 250 mg to about 700 mg, or from about 700 mg to about 1500 mg. The tablet can have any of a variety of shapes, including traditional pill or tablet shapes.

Generally, the product in tablet form comprises a glucose-polysaccharide blend and a sugar alcohol. In some embodiments, the glucose-polysaccharide blend is present in an amount of from about 35 to about 50% by weight, based on the total weight of the product; and the sugar alcohol is present in an amount of from about 30 to about 45% by weight, based on the total weight of the product. In some embodiments, the sugar alcohol is isomalt, erythritol, sorbitol, arabitol, ribitol, maltitol, dulcitol, iditol, mannitol, xylitol, lactitol, or a combination thereof. In some embodiments, the sugar alcohol is or comprises isomalt.

When in the form of a tablet, the product may be dissolvable. As used herein, the terms “dissolve,” “dissolving,” and “dissolvable” refer to products having aqueous-soluble components that interact with moisture in the oral cavity and enter into solution, thereby causing gradual consumption of the product. According to one aspect, the dissolvable product is capable of lasting in the user's mouth for a given period of time until it completely dissolves. Dissolution rates can vary over a wide range, from about 1 minute or less to about 60 minutes. For example, fast release products typically dissolve and/or release the desired component(s) (e.g., active ingredient, flavour, and the like) in about 2 minutes or less, often about 1 minute or less (e.g., about 50 seconds or less, about 40 seconds or less, about 30 seconds or less, or about 20 seconds or less). Dissolution can occur by any means, such as melting, mechanical disruption (e.g., chewing), enzymatic or other chemical degradation, or by disruption of the interaction between the components of the product. In other embodiments, the products do not dissolve during the product's residence in the user's mouth.

### Lozenge

In some embodiments, the products disclosed herein may be in the form of a dissolvable lozenge product configured for oral use. Example lozenge-type products of the invention have the form of a lozenge, tablet, microtab, or other tablet-type product. See, for example, the types of nicotine-containing lozenges, lozenge formulations, lozenge formats and configurations, lozenge characteristics and techniques for formulating or manufacturing lozenges set forth in U.S. Pat. No. 4,967,773 to Shaw; U.S. Pat. No. 5,110,605 to Acharya; U.S. Pat. No. 5,733,574 to Dam; U.S. Pat. No. 6,280,761 to Santus; U.S. Pat. No. 6,676,959 to Andersson et al.; U.S. Pat. No. 6,248,760 to Wilhelmsen; and U.S. Pat. No. 7,374,779; US Pat. Pub. Nos. 2001/0016593 to Wilhelmsen; 2004/0101543 to Liu et al.; 2006/0120974 to Mcneight; 2008/0020050 to Chau et al.; 2009/0081291 to Gin et al.; and 2010/0004294 to Axelsson et al.; which are incorporated herein by reference.

Lozenge products are generally described as “hard”, and are distinguished in this manner from soft lozenges (i.e., pastilles). Hard lozenges are mixtures of sugars and/or carbohydrates in an amorphous state. Although they are made from aqueous syrups, the water, which is initially present, evaporates as the syrup is boiled during processing so that the moisture content in the finished product is very low, such as 0.5% to 1.5% by weight. To obtain lozenges that are hard and not tacky, the temperature of the melt generally must reach the hard crack stage, with an example temperature range of 149° to 154° C.

Lozenge-type products, in some embodiments, may exhibit translucence or transparency. The desired transparency or translucency of the product can be quantified by any known method. For example, optical methods such as turbidimetry (or nephelometry) and colorimetry may be used to quantify the cloudiness (light scattering) and the color (light absorption), respectively, of the products. Translucency can also be confirmed by visual inspection by simply holding the product up to a light source and determining if light travels through the material or product in a diffuse manner.

The lozenge-type products of the present disclosure may incorporate various different additives in addition to the combination of active ingredients, and may be prepared according to a variety of different methods commonly known in the art for preparing lozenge-type products. Example compositions, products, and methods of preparing such products will be detailed herein below.

Lozenge products of the present disclosure typically include a composition comprising the combination of active ingredients in an amount of less than about 2% by weight, a sugar substitute in an amount of at least about 80% by weight, and a sugar alcohol syrup. Any active ingredient as discussed herein is suitable for use as an active ingredient in the lozenge products provided herein. In some embodiments, the active ingredient may be provided in liquid form or in a dry powder or particulate form. As noted above, the active ingredient typically is present in an amount from about 0.1% to about 10% by weight, such as, e.g., from about 0.1% to about 10% by weight, such as, e.g., from about 0.1%, about 0.5%, about 1%, about 1.5%, about 2%, about 2.5%, about 3%, about 3.5%, about 4%, or about 4.5% by weight, to about 5.5%, about 6%, about 6.5%, about 7%, about 7.5%, about 8%, about 8.5%, about 9%, about 9.5%, or about 10% by weight, based on the total weight of the product. In some embodiments, the active ingredient may be present in an amount of less than about 10%, less than about 9%, less than about 8%, less than about 7%, less than about 6%, less than about 5%, less than about 4%, less than about 3%, less than about 2%, or less than about 1% by weight, based on the total weight of the product.

In some embodiments, the lozenge product comprises a sugar substitute. The sugar substitute is typically provided in pure, solid form (e.g., granular or powdered form). In certain embodiments, the sugar substitute is dry, comprising a very low water content. For example, the sugar substitute can comprise less than about 5% water by weight, less than about 3% water by weight, less than about 2% water by weight, or less than about 1% water by weight. In certain embodiments, the sugar substitute is capable of forming a glassy matrix. The formation of a glassy matrix is commonly characterized by a translucent/transparent appearance.

Typically, the sugar substitute is substantially non-hygroscopic. Non-hygroscopic materials typically do not absorb, adsorb, and/or retain a significant quantity of moisture from the air. Non-hygroscopic materials can provide the benefit of reducing the tendency of the lozenge product to tackify upon exposure to humidity. The sugar substitute can be any sugarless material (i.e., sucrose-free material) and can be natural or synthetically produced. The sugar substitute used in the products described herein can be nutritive or non-nutritive. For example, the sugar substitute is commonly a sugar alcohol. Sugar alcohols that may be useful according to the present invention include, but are not limited to, erythritol, threitol, arabitol, xylitol, ribotol, mannitol, sorbitol, dulcitol, iditol, isomalt, maltitol, lactitol, polyglycitol, and mixtures thereof. For example, in certain embodiments, the sugar alcohol is selected from the group consisting of erythritol, sorbitol, and isomalt. The amount of sugar substitute in the lozenge products can vary, but is typically at least about 75%, at least about 80%, at least about 85%, or at least about 90%, or at least about 95% by weight of the product.

In certain embodiments, the sugar substitute comprises one or more sugar alcohols. For example, in one embodiment, the sugar substitute is isomalt.

In some embodiments, the sugar substitute is one or more of allulose, soluble tapioca fiber, and inulin. Such sugar substitutes may be an alternative to sugar alcohols, or used in combination with one or more sugar alcohols.

In some embodiments, the lozenge products of the present disclosure may comprise a syrup, e.g., a sugar syrup or a sugar alcohol syrup. “Sugar alcohol syrup” as used herein is intended to refer to a thick solution of sugar alcohol in water, e.g., having greater than about 40% solids, preferably having greater than about 50% solids, greater than about 60% solids, greater than about 70% solids, or greater than about 80% solids. Typically, the solid content of the sugar alcohol syrup primarily comprises the named sugar alcohol (i.e., maltitol syrup typically comprises greater than about 80%, greater than about 85%, or greater than about 90% by weight maltitol on a dry basis). Sugar alcohol syrups are generally prepared by heating a solution of the sugar alcohol in water and cooling the mixture to give a viscous composition. The resulting syrup is typically characterized by a relatively high concentration of sugar alcohol and relatively high stability (i.e., the sugar alcohol typically does not crystallise from solution, e.g., at room temperature).

The syrup, e.g., sugar alcohol syrup, desirably is capable of affecting the recrystallsation of a melted sugar substitute. One example sugar alcohol syrup that is particularly useful according to the present disclosure is maltitol syrup. Other sugar alcohol syrups can be used, including, but not limited to, com syrup, golden syrup, molasses, xylitol, mannitol, glycerol, erythritol, threitol, arabitol, ribitol, mannitol, sorbitol, dulcitol, iditol, isomalt, lactitol, and polyglycitol syrups. Such sugar alcohol syrups can be prepared or can be obtained from commercial sources. For example, maltitol syrups are commercially available from such suppliers as Corn Products Specialty Ingredients. Although sugar alcohol syrups may be preferred, sugar syrups can, in certain embodiments, be used in place of or in combination with the sugar alcohol syrup. For example, in some embodiments, corn syrup, golden syrup, and/or molasses can be used.

The amount of sugar alcohol syrup added to the lozenge composition mixture is typically that amount required to slow recrystallisation of the sugar substitute in melted form. It should be noted that it may be possible to vary the amount of sugar alcohol syrup depending on the composition of the remaining ingredients to ensure that the recrystallisation is sufficiently slow to provide a material with the desired characteristics (e.g., a desired level of translucency/transparency). Accordingly, the amount of sugar alcohol syrup can vary, but typically ranges from about 0.1% to about 2%, often from about 0.5% to about 1.5%, and more often about 1% by weight of the lozenge product mixture. In certain embodiments, the amount of sugar alcohol syrup is higher, for example, up to about 2% by weight of the mixture, up to about 5% by weight of the mixture, up to about 10% by weight of the mixture, or up to about 20% by weight of the mixture.

Representative lozenge compositions and products may incorporate about 10% by weight or less of the combination of active ingredients, about 0.01 to about 2% artificial sweetener, about 1% to about 5% humectant, about 1% to about 5% natural sweetener, at least about 80% of a sugar substitute, about 0.1% to about 10% of a sugar alcohol syrup, one or more flavorants in an amount of up to about 5%, and salt in an amount up to about 3%, based on the total weight of the product. The particular percentages and choice of ingredients will vary depending upon the desired flavor, texture, and other characteristics.

Oral products of the present disclosure in the form of a lozenge may contain various amounts of water. The water content of the lozenge described herein, prior to use by a consumer of the product, may vary within such ranges according to the desired properties and characteristics, in addition to dictating the final form of the product. For example, lozenge-type products typically possess a water content in the range of about 0.1 to about 5% by weight of the product. Preferably, the moisture content of a lozenge product, as present within a single unit of product prior to insertion into the mouth of the user, is less than about 5%, less than about 3%, less than about 2%, or less than about 1% by weight of the product. In some embodiments, the moisture content of a lozenge product as described herein may be within the range of about 0.1% to about 5%, about 0.5 to about 3%, or about 1 to about 2% by weight of the product.

### Powders or Pouched Products

In some embodiments, the oral product may be in the form of a powder. The powder may be a free-flowing powder. The powder may be contained in loose form within a container, and may thus be used in a form similar to tobacco snuff where the user takes a pinch of powder from the container and places the powder in the oral cavity. Alternatively or additionally, the powder may be incorporated into a moisture-permeable (e.g. saliva-permeable) pouch, similar to a snus-type product. The pouched product may be configured for insertion into the oral cavity of a user; i.e. it may be a pouched oral product.

In some embodiments, the product of the present disclosure is in the form of a pouched oral product. Such a pouched product comprises the oral product as described herein, disposed within a moisture-permeable container (e.g., a water-permeable pouch or saliva-permeable pouch). For example, the pouched product may comprise the oral product in a powder form incorporated within the saliva-permeable pouch.

Such compositions in the moisture-permeable pouch format are typically used by placing one pouch containing the composition in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the composition therein (e.g., the active ingredients and/or any flavours) to pass through e.g., the moisture-permeable pouch and provide the user with flavour and satisfaction, and the user is not required to spit out any portion of the composition. After about 10 minutes to about 60 minutes, typically about 15 minutes to about 45 minutes, of use/enjoyment, substantial amounts of the composition have been ingested by the human subject, and the pouch may be removed from the mouth of the human subject for disposal.

In some embodiments, the pouch is saliva-permeable. This means that the pouch is made of a saliva-permeable pouch material. The pouch material used in oral pouched products is typically a dry-laid bonded nonwoven comprising viscose rayon fibres (i.e. regenerated cellulose) and an acrylic polymer that acts as binder in the nonwoven material and provides for heat-sealing of the pouches during manufacturing thereof. The pouch material may also comprise synthetic fibres (e.g. polyester) in addition to viscose fibres. The viscose nonwoven material normally used for smokeless tobacco pouches is similar to the fabric used in tea bags. Nonwovens are fabrics that are neither woven nor knitted. Methods for the manufacturing of nonwoven materials are commonly known in the art. Further information on nonwovens is found in “Handbook of Nonwovens” by S. Russel, published by Woodhead Pub I. Ltd., 2007. In some embodiments, the pouch material is a fleece material. In some embodiments, the pouch material is a non-woven material. In some embodiments, the pouch material is a non-woven fleece material. In some embodiments, the pouch material comprises viscose, such as viscose rayon fibres. In some embodiments, the pouch material comprises regenerated cellulose fibres. In some embodiments, the pouch material comprises polyester fibres; the polyester fibres may constitute the pouch material or may be included in combination with viscose (such as regenerated cellulose fibres).

In some embodiments, the pouch material comprises a binder that provides for heat sealing of the pouches during manufacture. In some embodiments, the pouch material comprises an acrylic binder. In some embodiments, the pouch material comprises an acrylic binder in combination with viscose and/or polyester fibres.

Suitable packets, pouches or containers of the type used for the manufacture of smokeless tobacco products are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. The composition may be contained in pouches and packaged, in a manner and using the types of components used for the manufacture of conventional snus types of products. The pouch provides a moisture-permeable container of a type that may be considered to be similar in character to the mesh-like type of material that is used for the construction of a tea bag. Components of the composition readily diffuse through the pouch and into the mouth of the user. Non-limiting examples of suitable types of pouches are set forth in, for example, U.S. Pat. No. 5,167,244 to Kjerstad and U.S. Pat. No. 8,931,493 to Sebastian et al.; as well as US Patent App. Pub. Nos. 2016/0000140 to Sebastian et al.; 2016/0073689 to Sebastian et al.; 2016/0157515 to Chapman et al.; and 2016/0192703 to Sebastian et al., each of which is incorporated herein by reference. Pouches can be provided as individual pouches, or a plurality of pouches (e.g., 2, 4, 5, 10, 12, 15, 20, 25 or 30 pouches) can be connected or linked together (e.g., in an end-to-end manner) such that a single pouch or individual portion can be readily removed for use from a one-piece strand or matrix of pouches.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and composition each may undergo complete dispersion within the mouth of the user during normal conditions of use, and hence the pouch and composition both may be ingested by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). Preferred pouch materials, though water dispersible or dissolvable, may be designed and manufactured such that under conditions of normal use, a significant amount of the composition contents permeate through the pouch material prior to the time that the pouch undergoes loss of its physical integrity. If desired, flavoring ingredients, disintegration aids, and other desired components, may be incorporated within, or applied to, the pouch material.

The amount of the oral product contained within each pouched product unit, for example, a pouch, may vary. In some embodiments, the weight of the composition within each pouch is at least about 50 mg, for example, from about 50 mg to about 1 gram (1,000 mg), such as from about 100 mg to about 900 mg, such as from about 200 mg to about 800 mg, such as from about 500 mg to about 700 mg. In some smaller embodiments, the weight of the composition within each pouch may be from about 100 mg to about 300 mg. For a larger embodiment, the weight of the composition within each pouch may be from about 300 mg to about 700 mg. If desired, other components can be contained within each pouch.

The moisture content of the oral product may vary depending on the format in which the composition is provided. In some embodiments as described hereinabove, the oral product may be in the form of moist snuff or snus and/or may be provided in pouched formats. In some embodiments (e.g. for snus-type products), the moisture content of the composition (before insertion of the product into the user's mouth) may be at least about 20% by weight, such as at least 30% by weight, such as at least 40% by weight, such as at least 50% by weight of the oral product. In some embodiments (for example, for snus-type products; e.g. non-pouched or pouched snus products), the moisture content of the composition (before insertion of the product into the user's mouth) may be from about 20% to about 70% by weight, such as from about 30% to about 60% by weight, such as from about 40% to about 55% by weight of the oral product.

In some embodiments, the oral product may be a snus-type or snuff-type product that is in ‘dry’ form. In such embodiments, the moisture content of the oral product may be no greater than about 10% by weight, such as no greater than about 5% by weight of the oral product. For example, the moisture content may be from about 0.1% to about 10% by weight, such as from about 1% to about 5% by weight of the oral product.

When in the form of a pouched oral product, the oral product typically comprises a filler. The filler may preferably be a cellulose material selected from the suitable materials described hereinabove. In some preferred embodiments, the filler is or comprises at least MCC. The amount of filler can vary, but is typically at least about 5% to about 95% by weight of the oral product, based on the total weight of the oral product. In some embodiments, the filler (such as a cellulose material, such as MCC) may be present in the oral product in an amount of from about 5% to about 95% by weight, such as from about 10% to about 90% by weight, such as from about 15% to about 85% by weight, such as from about 20% to about 80% by weight, such as from about 25% to about 75% by weight, such as from about 30% to about 70% by weight, such as from about 35% to about 65% by weight, such as from about 40% to about 60% by weight of the oral product. In some embodiments, the filler (such as a cellulose material, such as MCC) may be present in an amount of from about 45% to about 55% by weight of the oral product.

### Package

According to some embodiments described herein, there is provided a package containing an oral product as described herein. For example, the package may contain the oral product in powdered form. In such embodiments, the package may be in the form of a tin or plastic container. Alternatively or additionally, the package may contain the oral product in the form of lozenge, tablet, or the like. The package may be in the form of a blister pack, tin or plastic container containing such oral dosage forms.

According to some embodiments described herein, there is provided a package containing at least one pouched oral product as described herein. A pouched product as described herein can be packaged within any suitable inner packaging material and/or outer container. See also, for example, the various types of containers for smokeless types of products that are set forth in U.S. Pat. No. 7,014,039 to Henson et al.; U.S. Pat. No. 7,537,110 to Kutsch et al.; U.S. Pat. No. 7,584,843 to Kutsch et al.; U.S. Pat. No. 8,397,945 to Gelardi et al., D592,956 to Thiellier; D594, 154 to Patel et al.; and D625,178 to Bailey et al.; US Pat. Pub. Nos. 2008/0173317 to Robinson et al.; 2009/0014343 to Clark et al.; 2009/0014450 to Bjorkholm; 2009/0250360 to Bellamah et al.; 2009/0266837 to Gelardi et al.; 2009/0223989 to Gelardi; 2009/0230003 to Thiellier; 2010/0084424 to Gelardi; and 2010/0133140 to Bailey et al; 2010/0264157 to Bailey et al.; and 2011/0168712 to Bailey et al. which are incorporated herein by reference. For example, the package may be a tin or plastic container which contains a plurality of the pouched oral products.

In some preferred embodiments in which the oral product is in the form of a liquid, there is provided a package in the form of a bottle or can that contains the liquid oral dosage form. The package may be a bottle containing the desired volume of liquid oral dosage form.

### Process

The manner by which the various components of the composition (e.g., active ingredients and any additives) are combined may vary. As such, the overall product with e.g., powdered composition components may be relatively uniform in nature (e.g., homogenous). The components noted above, which may be in liquid or dry solid form, can be admixed in a pre-treatment step prior to mixture with any remaining components of the product, or simply mixed together with all other liquid or dry ingredients.

The various components of the product may be contacted, combined, or mixed together using any mixing technique or equipment known in the art. Any mixing method that brings the product ingredients into intimate contact can be used, such as a mixing apparatus featuring an impeller or other structure capable of agitation. Examples of mixing equipment include casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. In some embodiments, the components forming the product are prepared such that the mixture thereof may be used in a starch molding process for forming the product. Manners and methods for formulating products will be apparent to those skilled in the art. See, for example, the types of methodologies set forth in U.S. Pat. No. 4,148,325 to Solomon et al.; U.S. Pat. No. 6,510,855 to Korte et al.; and U.S. Pat. No. 6,834,654 to Williams, U.S. Pat. No. 4,725,440 to Ridgway et al., and 6,077,524 to Bolder et al., each of which is incorporated herein by reference.

### Method of Preparing a Liquid Oral Dosage Form

In accordance with some embodiments described herein, there is provided a process for preparing an oral product as described herein, the process comprising the steps of:
(a) combining active ingredients,
(b) contacting the active ingredients with water, and
(c) mixing the active ingredients with water to prepare an oral product.

The combination of active ingredients may be as described hereinabove. The combination of actives may also be as described below in respect of “Further Broad Aspects”.

The active ingredients may be provided in the form of a liquid extract, a liquid oil or a powder. When in the form of a powder, step (c) may comprise mixing the active ingredients with water until said active ingredients have dissolved in the water.

Step (b) and/or step (c) may preferably be carried out at ambient or room temperature (20-25° C.). Alternatively, the temperature may be elevated in order to assist with dispersion or dissolution of the active ingredients in the water. For example, step (b) may comprise contacting the active ingredients with water at a temperature of from about 20-100° C., such as from about 30-90° C., or from about 40-80° C. Step (b) may comprise mixing the active ingredients with water at a temperature of from about 20-100° C., such as from about 30-90° C., or from about 40-80° C.

The process may comprise adding any additional additives at any stage. For example, any additives may be added to the active ingredients prior to combination with water and/or after the active ingredients have been combined with water. The additives may also be added to the water prior to contacting the water with the combination of active ingredients. Step (a) may comprise an optional step of combining any additives (e.g. acidifying agent) with the combination of active ingredients. Step (b) may comprise an optional step of contacting the active ingredients with water and an additive. Step (c) may comprise an optional step of adding additives to the mixture and mixing the additives with the combination of active ingredients and water.

In some embodiments, the oral product further comprises one or more additives selected from a thickener, an organic acid, or mixtures thereof. In such embodiments, the one or more additives may be combined with water prior to addition of the active ingredients. Optionally, the step of combining the one or more additives with water may comprise heating the combination, e.g. to a temperature of from about 60° C. to about 80° C., to achieve dissolution of the additive(s). Further additives, such as a sweetener, humectant, colouring agent or the like, may be added at this stage or may be added during and/or after the step of combining the active ingredients with the water.

The resulting liquid product may be in the form of a solution or dispersion of active ingredients in water.

### Method of Preparing Tablet Products

In some embodiments, the product is in the form of a compressed pellet or tablet. In one embodiment, the process for making the pellet or tablet involves first mixing the bulk filler (e.g., EMDEX®) and the active ingredients. The remaining composition ingredients (e.g., sugar alcohol and any other desired components, such as binders, colorants, sweeteners, flavors, and the like) are then added. Optionally, a colorant can may be added to one of the composition components in a separate step prior to mixing with the remaining components of the composition. The mixing of the composition can be accomplished using any mixing device. The final composition is then compressed into pellet or tablet form using conventional tableting techniques and optionally coated. Compressed composition pellets can be produced by compacting the composition, including any associated formulation components, in the form of a pellet, and optionally coating each pellet with an overcoat material. Example compaction devices, such as compaction presses, are available as Colton 2216 and Colton 2247 from Vector Corporation and as 1200i, 2200i, 3200, 2090, 3090 and 4090 from Fette Compacting. Devices for providing outer coating layers to compacted pelletized compositions are available as CompuLab 24, CompuLab 36, Accela-Cota 48 and Accela-Cota 60 from Thomas Engineering.

When present, a coating typically comprises a film-forming polymer, such as a cellulosic polymer, an optional plasticizer, and optional flavorants, colorants, salts, sweeteners or other additives of the types set forth herein. The coating compositions are usually aqueous in nature and can be applied using any pellet or tablet coating technique known in the art, such as pan coating. Example film-forming polymers include cellulosic polymers such as methylcellulose, hydroxypropyl cellulose (HPC), hydroxypropyl methylcellulose (HPMC), hydroxyethyl cellulose, and carboxy methylcellulose. Example plasticizers include aqueous solutions or emulsions of glyceryl monostearate and triethyl citrate. Additional potential coatings include food grade shellac, waxes such as carnuaba wax, and combinations thereof.

### Methods of Preparing Lozenge Products

The manners and methods used to formulate and manufacture a lozenge product as described herein above can vary. For example, the compositions can be prepared via any method commonly used for the preparation of hard boiled confections. Example methods for the preparation of hard confections can be found, for example, in LFRA Ingredients Handbook, Sweeteners, Janet M. Dalzell, Ed., Leatherhead Food RA (December 1996), pp. 21-44, which is incorporated herein by reference.

Typically, a first mixture of ingredients is prepared. The composition of the first mixture of ingredients can vary; however, it typically comprises a sugar substitute and may contain various additional substances (e.g., the sugar alcohol syrup, NaCl, preservatives, further sweeteners, water, and/or flavourings). In certain embodiments, it comprises the sugar substitute, salt, and vanillin. In other embodiments, the first mixture comprises the sugar substitute and the sugar alcohol syrup. Typically, the first mixture of ingredients does not contain the active ingredients; although, it some embodiments, the active ingredients may be incorporated into the first mixture of ingredients.

The first mixture of ingredients is heated until it melts; subsequently, the mixture is heated to or past the hard crack stage. In confectionary making, the hard crack stage is defined as the temperature at which threads of the heated mixture (obtained by pulling a sample of cooled syrup between the thumb and forefinger) are brittle or as the temperature at which trying to mold the syrup results in cracking. According to the present method, the temperature at which the hard crack stage is achieved can vary, depending on the specific makeup of the product mixture but generally is between about 145° C. and about 170° C. Typically, the mixture is not heated above about 171° C., which is the temperature at which caramelisation begins to occur. In the processes of the present disclosure, the mixture is typically heated to the hard crack stage temperature or above and then allowed to cool. The heating can be conducted at atmospheric pressure or under vacuum. Typically, the method of the present invention is conducted at atmospheric pressure.

In one example embodiment, the first mixture of ingredients comprises a high percentage of isomalt and the mixture is heated to about 143° C. Once all components are dissolved, the temperature is raised past the hard crack stage (e.g., to about 166° C.). The mixture is heated to this temperature and then removed from the heat to allow the mixture to cool.

In certain embodiments, the active ingredients and, optionally, additional components (e.g., additional sweeteners, fillers, flavouring agents, and water) as described above are separately combined in a second mixture. The second mixture is added to the first mixture of ingredients, typically after the first mixture of ingredients has been removed from the heat. The addition of the second mixture may, in some embodiments, occur only after the heated first mixture of ingredients has cooled to a predetermined temperature (e.g., in certain embodiments, to about 132° C.). In certain embodiments, one or more flavouring agents are added to the second mixture immediately prior to adding the mixture to the first, heated mixture of ingredients. Certain flavouring agents are volatile and are thus preferably added after the mixture has cooled somewhat. The combined mixture is then formed into the desired shape. In certain embodiments, the mixture is poured directly into molds, formed (e.g., rolled or pressed) into the desired shape, or extruded. If desired, the mixture can be extruded or injection molded. In certain embodiments, the mixture is formed or extruded into a mold of desired shape in an enclosed system, which may require decreased temperature and which may limit evaporation of certain mixture components. For example, such a system may limit the evaporation of volatile components including, but not limited to, flavorants. Other methods of producing lozenges are also intended to be encompassed herein.

Typical conditions associated with manufacture of food-grade lozenge products such as described herein include control of heat and temperature (i.e., the degree of heat to which the various ingredients are exposed during manufacture and the temperature of the manufacturing environment), moisture content (e.g., the degree of moisture present within individual ingredients and within the final composition), humidity within the manufacturing environment, atmospheric control (e.g., nitrogen atmosphere), airflow experienced by the various ingredients during the manufacturing process, and other similar types of factors. Additionally, various process steps involved in product manufacture can involve selection of certain solvents and processing aids, use of heat and radiation, refrigeration and cryogenic conditions, ingredient mixing rates, and the like. The manufacturing conditions also can be controlled due to selection of the form of various ingredients (e.g., solid, liquid, or gas), particle size or crystalline nature of ingredients of solid form, concentration of ingredients in liquid form, or the like. Ingredients can be processed into the desired composition by techniques such as extrusion, compression, spraying, and the like.

In certain embodiments, the lozenge product may be transparent or translucent. As used herein, “translucent” or “translucency” refers to materials allowing some level of light to travel therethrough diffusely. In certain embodiments, lozenge products of the present disclosure can have such a high degree of clarity that the material can be classified as “transparent” or exhibiting “transparency,” which is defined as a material allowing light to pass freely through without significant diffusion. The clarity of the lozenge product is such that there is some level of translucency as opposed to opacity (which refers to materials that are impenetrable by light).

Transparency/translucency can be determined by any means commonly used in the art; however, it is commonly measured by spectrophotometric light transmission over a range of wavelengths (e.g., from about 400-700 nm). Alternatively, optical methods such as turbidimetry (or nephelometry) and colorimetry may be used to quantify the cloudiness (light scattering) and the color (light absorption), respectively, of the lozenge products provided herein. Translucency can also be confirmed by visual inspection by simply holding the material (e.g., extract) or product up to a light source and determining if light travels through the product in a diffuse manner.

### Method of Preparing Melt Products

In some embodiments, the product is in meltable form. For preparation of meltable products, the lipid is typically heated to slightly above the melting temperature such that the lipid is liquefied. Optionally, active ingredients, flavoring agents, and/or lecithin can be added to the liquefied lipid at this stage. Thereafter, all or a portion of the liquefied lipid can be blended with the dry blend and mixed until the product reaches the desired level of homogeneity or until the desired textural properties are achieved. The mixture is milled (e.g., in a dry roll mill) until the particle size is less than about 20 microns. The milled isomalt-palm oil is combined with any remaining lipid, and the dry ingredients and flavor mixed in. The base is generally warmed to a fluid consistency.

In some embodiments, a sugar alcohol (e.g., isomalt) is added to a mixer bowl, and a portion of the total lipid (e.g., melted palm oil) is added, along with salt and emulsifier.

Additional lipid is added with mixing until adhesive clumps form. The clumped mixture is transferred portion-wise to a 3 roll mill and processed to a particle size of less than 50 microns, or about 20 microns. The refined mixture is transferred to a mixer bowl, and the remaining lipid added with mixing. The mixture is warmed as necessary to maintain a fluid consistency. Sweetener, flavor, and active ingredient(s) are added with mixing. Mixing is continued until a homogenous composition is obtained. The mixture is allowed to rest for a period of time, such as about 10 to 15 minutes. The composition can be divided into discrete portions, such as by pouring the composition into a sheet-like structure, cooling, and then cutting the structure into individual portions, or by depositing the composition into molds and allowing to cool. The molds may be starch molds or starchless molds. In particular embodiments, the molds are starchless.

The melt composition may be held in the mold (starch or starchless) for a predetermined duration of time such as, for example, from about 1 to about 15 minutes, to allow the melt composition to cool and solidify. Optionally, the molds containing the melt product may be cooled by refrigeration to accelerate solidification.

According to other aspects of the present disclosure, rather than using molds to prepare the melt product, an extrusion process may be employed in which the final melt product is extruded. In some instances, the melt composition in slurry form may be formed into a sheet and allowed to dry to a moisture content, for example, of about 15 percent to about 25 percent by weight water to form a tacky or otherwise pasty material, which is in a form capable of physical handling. The material may then be chopped or otherwise cut into smaller pieces using, for example, a mixer. The chopped material may then be extruded through an extrusion device to any shape/size desired, including shapes that may be difficult or impossible to achieve with a mold. In some instances, the extruded product may then be dried to achieve a desired moisture content. A similar type process is described, for example, in U.S. Pat. No. 3,806,617 to Smylie et al., which is incorporated herein by reference in its entirety. Further, the melt composition may be subjected to a co-extrusion process with another composition.

Shapes such as, for example, rods and cubes can be formed by first extruding the material through a die having the desired cross-section (e.g., round or square) and then optionally cutting the extruded material into desired lengths. Techniques and equipment for extruding tobacco materials are set forth in U.S. Pat. No. 3,098,492 to Wursburg; U.S. Pat. No. 4,874,000 to Tamol et al.; 25 4,880,018 to Graves et al.; U.S. Pat. No. 4,989,620 to Keritsis et al.; U.S. Pat. No. 5,072,744 to Luke et al.; U.S. Pat. No. 5,829,453 to White et al.; and 6,182,670 to White et al.; each of which is incorporated herein by reference.

Example extrusion equipment suitable for use include food or gum extruders, or industrial pasta extruders such as Model TP 200/300 available from Emiliomiti, LLC of Italy. In some instances, a single machine may be capable of achieving multiple steps of the processes described herein, such as, for example, kneader systems available from Buss AG.

Although the foregoing description focuses on compositions that are uniform throughout each product unit, products can also be formed with multiple different formulations having different properties in the same product unit. For example, two different compositions can be deposited in a single mold to produce a layered product. Still further, two different compositions could be co-extruded to form a product with different characteristics across its cross-section. Such a process could be used to provide a product with two different compositions featuring different dissolution rates such that a first portion of the product dissolves at a first rate (e.g., a faster rate) and a second portion dissolves at a second, slower rate.

### Methods of Preparing Pouched Oral Products

Where the product is in the form of a pouched oral product, the process may comprise the steps of:
(a) combining active ingredients;
(b) contacting the combination of active ingredients and at least one filler to provide the oral product.

The combination of active ingredients may be as described hereinabove. The combination of actives may also be as described below in respect of “Further Broad Aspects”.

In some embodiments, the step (b) comprises mixing the at least one filler and the combination of active ingredients. In some embodiments, the combination of active ingredients is in solid form (e.g. in the form of a powder). The combination of active ingredients may be mixed directly with the filler to provide the oral product. In some embodiments, the combination of active ingredients may be dissolved in a hydrophilic solvent (e.g. water and/or alcohol) prior to contacting the filler. For example, the combination of active ingredients may be dissolved in water or alcohol (e.g. ethanol or propylene glycol) before being mixed with the filler. The process may, in such embodiments, comprise the step of drying the product so as to remove the solvent. For example, the product may be dried via heating, freeze-drying, spray-drying, or simply leaving the product at room temperature for a certain period of time. Preferably, the drying step comprises leaving the product at room temperature for a period of 1 hour to 48 hours to remove the solvent.

The process may then further comprise the step of pouching the oral product using a pouch material as described hereinabove.

### Use

According to some embodiments described herein, there is provided the use of a combination of active ingredients to increase alertness and/or energy levels of a human or animal, wherein the combination of active ingredients comprises (i) caffeine; (ii) a combination of B vitamins comprising vitamin B2, vitamin B3, vitamin B6, vitamin B9 and vitamin B12; and (iii) taurine.

According to other embodiments described herein, there is provided the use of a combination of active ingredients to increase alertness and/or energy levels of a human or animal, wherein the combination of active ingredients comprises (i) caffeine; (ii) a combination of B vitamins comprising vitamin B1, vitamin B2, vitamin B6, and vitamin B7; and (iii) taurine.

In some embodiments, the use comprises increasing or boosting the energy levels of the subject to which the combination of active ingredients is administered. The use may comprise increasing the alertness of the subject. In addition or alternatively, the use may comprise increasing the concentration and/or increasing the cognition of the subject.

According to some embodiments described herein, there is provided the use of a combination of active ingredients to increase the cognitive performance of a human or animal, wherein the combination of active ingredients comprises (i) caffeine; (ii) a combination of B vitamins comprising vitamin B2, vitamin B3, vitamin B6, vitamin B9 and vitamin B12; and (iii) taurine.

According to other embodiments described herein, there is provided the use of a combination of active ingredients to increase the cognitive performance of a human or animal, wherein the combination of active ingredients comprises (i) caffeine; (ii) a combination of B vitamins comprising vitamin B1, vitamin B2, vitamin B6, and vitamin B7; and (iii) taurine.

In some embodiments, the combination of active ingredients is as defined hereinabove. In such embodiments, the combination may further comprise any of the additional active ingredients as described hereinabove. For example, the combination of active ingredients may further comprise vitamin C, and/or ginseng. The combination of active ingredients may in particular further comprise ginseng.

Alternatively, the combination of active ingredients may be as described below in any of the “Further Broad Aspects”. For example, the combination of active ingredients may comprise (i) caffeine; (ii) ginseng; and (iii) taurine. Alternatively or in addition, the combination of active ingredients may comprise (i) caffeine; and (ii) taurine; wherein the caffeine and taurine are present in a weight ratio of about 1:1, and wherein the combination of active ingredients further comprises one or more of: (iii) at least one B vitamin selected from the group consisting of vitamin B1, vitamin B2, vitamin B3, vitamin B5, vitamin B6, vitamin B7, vitamin B9 and vitamin B12; or (iv) ginseng.

Alternatively or in addition, the combination of active ingredients may comprise (i) caffeine; (ii) a combination of B vitamins comprising vitamin B2, vitamin B3, vitamin B6, vitamin B9 and vitamin B12; (iii) ginseng. Alternatively or in addition, the combination of active ingredients may comprise (i) caffeine; (ii) at least one B vitamin selected from the group consisting of vitamin B2, vitamin B3, vitamin B6, vitamin B9 and vitamin B12; (iii) taurine; and (iv) vitamin C.

The combination of active ingredients may provide an improved effect in increasing the mental focus of the consumer as compared with previously known products. The present inventors have found that the specific combination of active ingredients of the present invention may improve alertness, and also increase physical and/or mental energy levels, and may also boost cognitive effects, such as reaction time and reduction in fatigue during cognitive tasks.

### Further Broad Aspects

In accordance with some embodiments described herein, there is provided an oral product comprising a combination of active ingredients, wherein the combination of active ingredients comprises (i) caffeine; (ii) ginseng and (iii) taurine.

The amounts of caffeine, taurine and ginseng (and all other active ingredients and/or additives) described hereinabove with respect to the first aspect are equally applicable to such embodiments comprising (i) caffeine, (ii) ginseng and (iii) taurine and are only not repeated here for conciseness. All amounts and combinations described hereinabove equally apply to this embodiment in which the inclusion of a combination of B vitamins is not essential.

In some preferred embodiments, the oral product comprising (i) caffeine, (ii) ginseng, and (iii) taurine is a liquid oral dosage form.

In some embodiments, the combination of active ingredients comprises (i) from about 0.1% to about 5% caffeine by weight of the oral product, (ii) from about 0.01% to about 1% ginseng by weight of the oral product, and (iii) from about 0.01% to about 5% taurine by weight of the oral product.

In some embodiments, the oral product comprises a combination of active ingredients comprising:
(i) caffeine, (ii) ginseng, and (iii) taurine,
wherein the weight ratio of caffeine to taurine is from about 5:1 to about 1:1;
wherein the weight ratio of caffeine to ginseng is from about 2:1 to about 1:1; and
wherein the weight ratio of taurine to ginseng is from about 2:1 to about 1:4.

Preferably, the weight ratio of caffeine to taurine is about 1:1.

In some embodiments, the oral product comprises a combination of active ingredients comprising:
(i) caffeine, (ii) ginseng, and (iii) taurine,
wherein the weight ratio of caffeine to taurine is about 1:1.

In some embodiments, the oral product comprises a combination of active ingredients comprising:
(i) caffeine, (ii) ginseng, and (iii) taurine,
wherein the weight ratio of caffeine to taurine is about 4:1.

In some embodiments, the oral product is a liquid oral dosage form comprising a combination of active ingredients comprising:
(i) caffeine, (ii) ginseng, and (iii) taurine,
wherein the weight ratio of caffeine to taurine is about 1:1 or about 4:1.

In some embodiments, the oral product is a liquid oral dosage form (e.g. in the form of a shot having a volume of 50 mL to 100 mL), where the liquid oral dosage form comprises:
(i) from about 50 mg to about 500 mg of caffeine;
(ii) from about 10 mg to about 300 mg of ginseng; and
(iii) from about 1 mg to about 500 mg taurine.

In some embodiments, the oral product is a liquid oral dosage form (e.g. in the form of a shot having a volume of 50 mL to 100 mL), where the liquid oral dosage form comprises:
(i) from about 100 mg to about 200 mg of caffeine;
(ii) from about 50 mg to about 200 mg of ginseng; and
(iii) from about 20 mg to about 200 mg taurine.

In any of the above embodiments, the oral product may further comprise an additional active ingredient as described hereinabove with respect to the first aspect. In particular, the oral product may further comprise at least one B vitamin and/or vitamin C. The ranges and combinations described hereinabove of these additional active ingredients equally apply to these embodiments. In some preferred embodiments, the oral product may further comprise at least one B vitamin selected from the group consisting of vitamin B1, B2, B3, B5, B6, B7, B9, B12 and combinations thereof. In some embodiments, the oral product may comprise a combination of at least vitamins B6, B9 and B12. Suitable ranges for amounts of these B vitamins is as described above.

The processes and uses described hereinabove may also equally apply to the above embodiments where the combination of active ingredients comprises: (i) caffeine; (ii) ginseng; and (iii) taurine.

In accordance with some embodiments described herein, there is provided an oral product in liquid form comprising a combination of active ingredients, wherein the combination of active ingredients comprises:
(i) caffeine;
(ii) a combination of B vitamins comprising vitamin B2, vitamin B3, vitamin B6, vitamin B9 and vitamin B12; and
(iii) ginseng.

The amounts of all active ingredients and/or additives described hereinabove with respect to the first aspect are equally applicable to such embodiments and are only not repeated here for conciseness.

In accordance with some embodiments described herein, there is provided an oral product comprising a combination of active ingredients, wherein the combination comprises: (i) caffeine; (ii) at least one B vitamin selected from the group consisting of vitamin B1, vitamin B2, vitamin B3, vitamin B5, vitamin B6, vitamin B7, vitamin B9 and vitamin B12; (iii) taurine; and (iv) vitamin C.

The amounts of caffeine, taurine, vitamin B and vitamin C (and all other active ingredients and/or additives) described hereinabove with respect to the first aspect are equally applicable to such embodiments and are only not repeated here for conciseness.

The processes and uses described hereinabove may also equally apply to the above embodiments.

All previously disclosed embodiments apply to these aspects as demonstrated by the appended claims.

### EXAMPLES

Aspects of the present invention are more fully illustrated by the following examples, which are set forth to illustrate certain aspects of the present invention and are not to be construed as limiting thereof.

### Example 1—Liquid Dosage Form

An oral product in the form of a liquid is prepared containing the following ingredients:

| Ingredients | Percentage amount (wt %) |
|---|---|
| Distilled water | 90-97 |
| Vitamins B2, B3, B6, B9, B12 | 0.01-0.5 |
| Caffeine | 0.1-1 |
| Taurine | 0.01-0.5 |
| Vitamin C | 0.1-2 |
| Ginseng extract | 0.1-1 |
| Citric acid | 0.1-1 |
| Sucralose | 0.01-1 |
| Thickener (e.g. xanthan gum) | 0.01-1 |
| Flavouring agent | 0.1-1 |
| Preservative (e.g. potassium sorbate) | 0.01-1 |

The oral product has a volume of 60 mL per serving.

The oral product is prepared by adding xanthan gum and citric acid to water under stirring. The mixture is heated to a temperature of between 60° C. and 80° C.

Once the xanthan gum and citric acid have dissolved, the mixture is allowed to cool to ambient temperature. All remaining ingredients are then added, and the resulting mixture stirred until a homogeneous dispersion or clear solution is obtained.

The product is then packaged in bottles, with each serving containing 60 mL of liquid.

### Example 2—Liquid Dosage Form Containing 4:1 Ratio of Caffeine:Taurine

An oral product in the form of a liquid is prepared containing the following ingredients:
Water
Vitamins B1, B2, B3, B5, B6, B7, B9, B12
Guarana extract (containing caffeine)—0.6% by weight
Taurine
Vitamin C
Ginseng extract—0.1% by weight
Citric acid
Sucralose
Gum Arabic
Thickener (xanthan gum)
Flavouring agent
Potassium sorbate

The amount of caffeine is about 120 mg and the amount of taurine is about 30 mg per serving. The amount of vitamin B6 is about 1.6 mg and the amount of vitamin B12 about 2 μg per serving. The oral product has a volume of 60 mL per serving.

The oral product is prepared as set out in Example 1.

### Example 3—Liquid Dosage Form

An oral product in the form of a liquid is prepared containing the following ingredients:
Water
Vitamin B1 (as thiamine mononitrate)—0.9 mg
Vitamin B2—0.98 mg
Vitamin B3 (as niacinamide)—12 mg
Vitamin B5 (as calcium pantothenate)—2.52 mg
Vitamin B6 (as pyridoxine hydrochloride)—1.7 mg
Vitamin B7—15.1 μg
Vitamin B9—340 μg
Vitamin B12—2.4 μg
Caffeine (green tea extract)—200 mg
Taurine
Vitamin C—90 mg
Ginseng root extract
Citric acid
Sucralose
Gum Arabic
Xanthan gum
Flavouring agent
Potassium sorbate

The total blend of caffeine, taurine and ginseng is present in an amount of 520 mg. The oral product has a volume of 60 mL per serving.

The oral product is prepared as set out in Example 1.

### Example 4—Results of Consumer Testing

A study was carried out to compare a product as prepared in Example 3 with a placebo (Kool-Aid). Blind testing was carried out where some test subjects consumed the shot of Example 3 while some were given Kool-Aid. The consumers were subjected to cognitively demanding tasks in the period immediately following consumption, with the below factors assessed over a period of up to 90 minutes post-consumption.

The following factors were assessed:
Cognitive accuracy—consumers were tested using Rapid Visual Information Processing (RVIP), which is the most demanding test within the cognitive battery. For RVIP, the results showed that the product of Example 3 outperformed the placebo, and maintained cognitive (RVIP) accuracy for up to 1.5 hours post-consumption during challenging tasks.
Mental arithmetic—mental arithmetic was assessed via timed and scored serial subtraction −3's. The oral product of Example 3 outperformed the placebo and demonstrated elevated performance over the time course of the demand battery (40-90 mins post-consumption).
Alertness—perceived effects were measured via a visual analogue scale (VAS): a 0-100 line from “least alert” to “most alert”. Perceived alertness for the oral product of Example 3 was significantly greater than the placebo, and was maintained over a period of 40-90 minutes post-consumption.
Physical energy—perceived effects were measured via a visual analogue scale (VAS): a 0-100 line from “least physically energised” to “most physically energised”. Perceived physical energy for the oral product of Example 3 was significantly greater than the placebo, and was maintained over a period of 40-90 minutes post-consumption.
Mental fatigue—users were subjected to cognitively demanding tasks, which were intended to cause fatigue. The consumers who drank the oral product of Example 3 gave limited self-reported fatigue (Profile of Mood States (POMS) main effects). The results showed that the oral product of Example 3 limits fatigue compared to the placebo over the 90 minutes duration of the study (VAS time course).

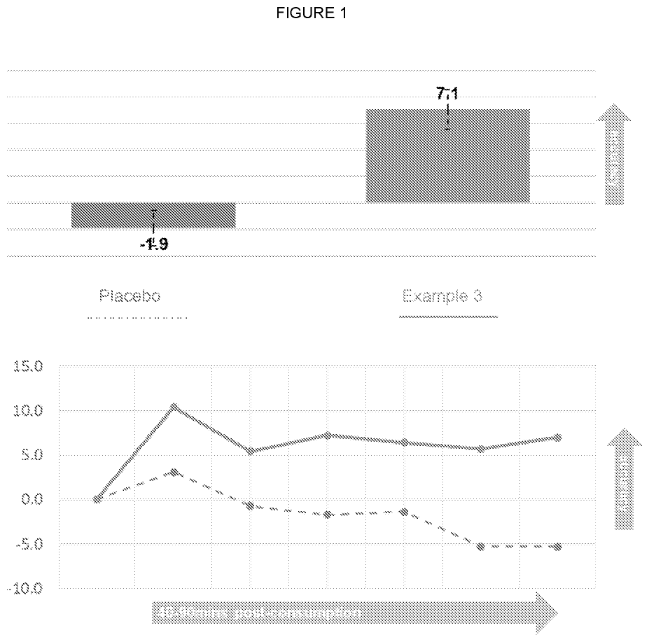

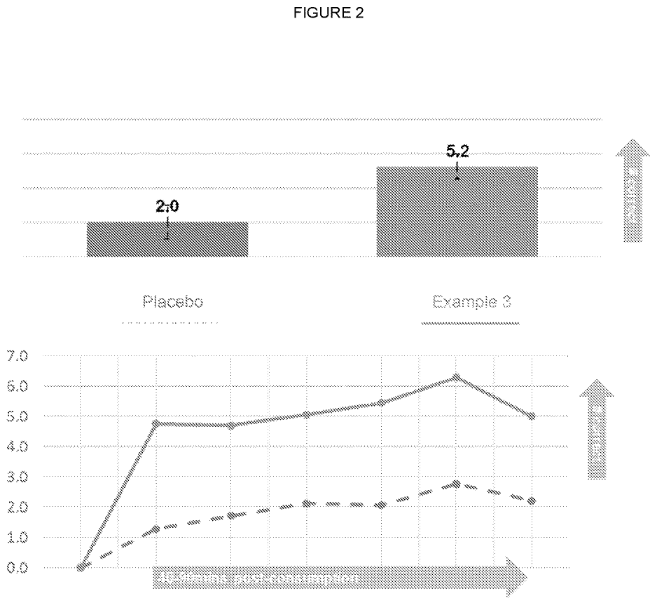

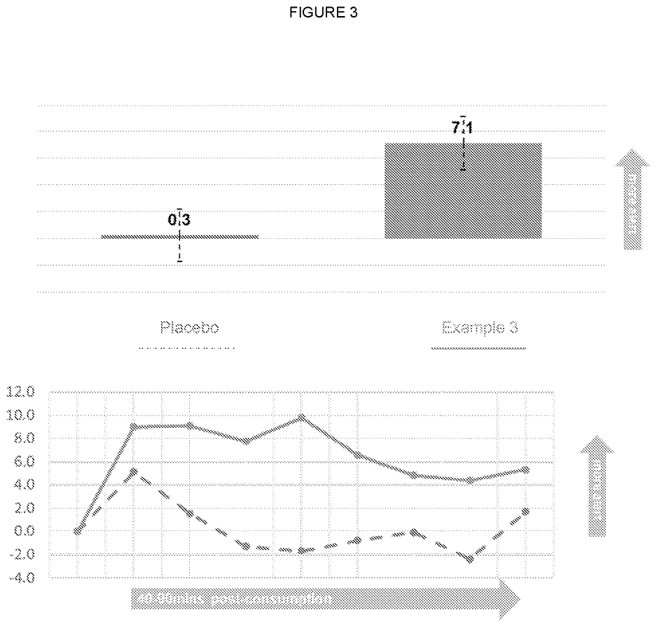

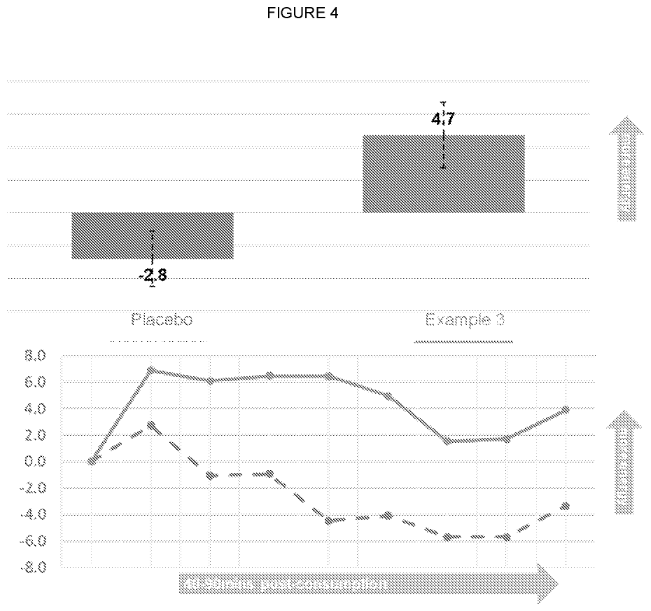

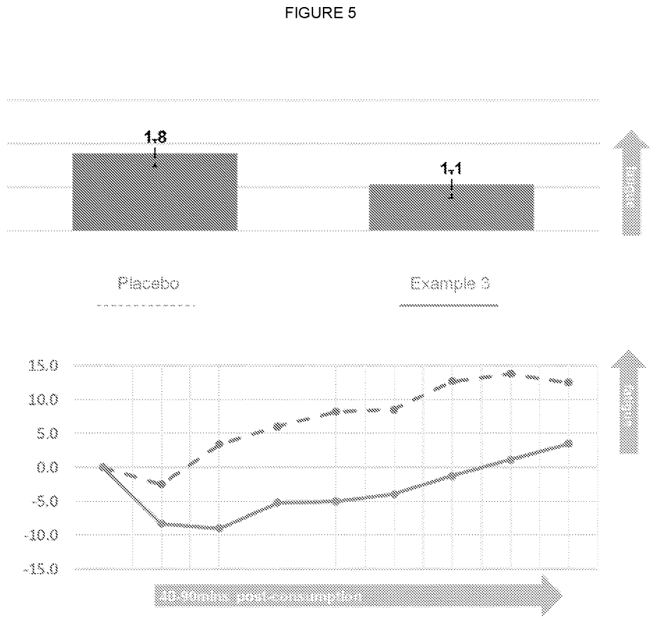

The results of the study are shown in FIGS. 1 to 5.

### Example 5

A study was carried out to compare a product as prepared in Example 3 with the following:
Placebo—Kool-Aid
Commercial product—5-Hour Energy, Berry flavour

The following factors were assessed:

| Endpoint | Method |
|---|---|
| Cognitive demand | COMPASS: |
| | RVIP |
| Executive function | Serial subtraction −7 |
| | Serial subtraction −3 |
| Attention and vigilance | NWM |
| | Corsi Blocks |
| Mood state | POMS |
| Pleasure, arousal, dominance | SAM |
| Fatigue; alertness; physical energy | VAS X7 |

The participants in the study were aged between 18-30 years old and were considered to be healthy adults. The participants refrained from physical activity and caffeine 10 hours prior to the testing, and any food and drink within 1 hour before testing. Cognitive battery tests were carried out before administration of any product (7X VAS/POMS/CATA, NWM, Corsi Block, RVIP, Serial −7 subtraction, Serial −3 subtraction). Each participant then consumed one of the oral products; the products were tested blind. After around 30 minutes after consumption, the participants were subjected to the same cognitive battery tests, which were repeated a number of times over a duration of up to 90 minutes after consumption.

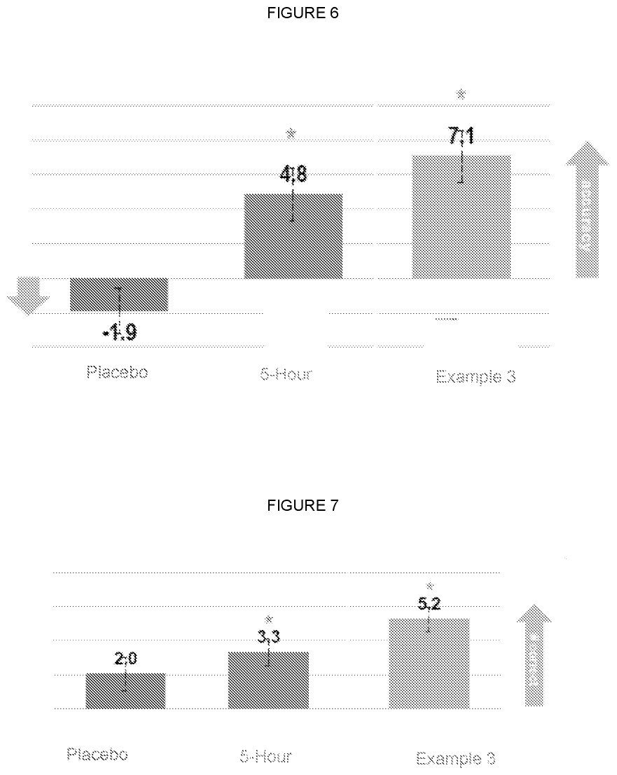

Cognitive accuracy, serial subtraction (−3) (number correct), alertness, physical energy and mental fatigue were measured using the same methodology as noted above for Example 4. The following results were found:
Cognitive accuracy—for main effects RVIP accuracy, both the oral product of Example 3 and 5-hour energy outperformed placebo, and maintained cognitive accuracy for up to 90 minutes post-consumption. Consumers were tested using Rapid Visual Information Processing (RVIP), which is the most demanding test within the cognitive battery. For RVIP. As shown in FIG. 6, Example 3 resulted in higher cognitive accuracy than 5-Hour Energy.
Mental arithmetic (Serial −3 subtraction)—the oral product of Example 3 outperformed both the placebo and 5-hour energy, and demonstrated elevated performance over the time course of the demand battery
Alertness (measured by VAS)—highest reported perceived alertness was shown for the product of Example 3.
Physical energy (measured by VAS)—highest reported perceived physical energy was shown for the product of Example 3.

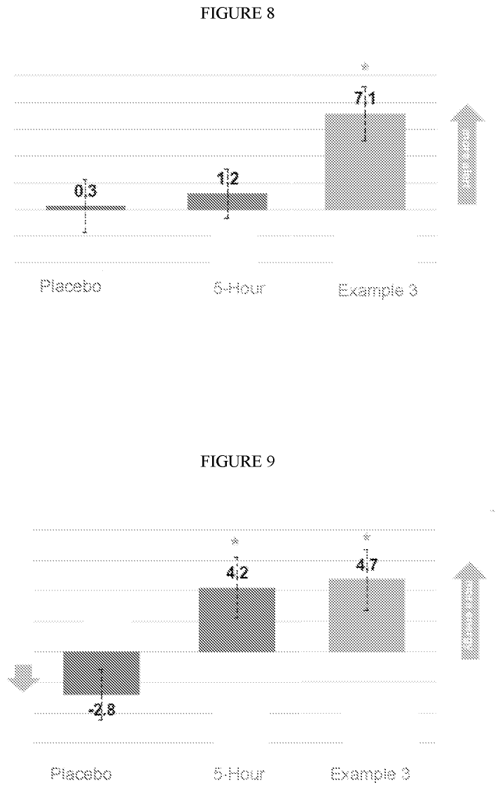

The results are shown in FIGS. 6 to 9.

### Example 6—Liquid Dosage Form

An oral product in the form of a liquid is prepared containing the following ingredients:
Water
Vitamin B1 (as thiamine mononitrate)—0.9 mg
Vitamin B2—0.98 mg
Vitamin B6 (as pyridoxine hydrochloride)—1.6 mg
Vitamin B7—15.1 μg
Caffeine—120 mg
Taurine
Vitamin C—24 mg
Ginseng root extract
Acidifier
Sweetener
Thickener
Flavouring agent
Preservative

The total blend of caffeine, taurine and ginseng is present in an amount of 290 mg. The oral product has a volume of 60 mL per serving.

The oral product is prepared as set out in Example 1.

### Example 7—Liquid Dosage Form

An oral product in the form of a liquid is prepared containing the following ingredients:
Water
Vitamin B1 (as thiamine mononitrate)—0.9 mg
Vitamin B2—0.98 mg
Vitamin B3 (nicotinamide)—12 mg
Vitamin B5 (as calcium pantothenate)—2.52 mg
Vitamin B6 (as pyridoxine hydrochloride)—1.7 mg
Vitamin B7—15.1 μg
Vitamin B9—200 μg
Vitamin B12—2.4 μg
Caffeine (green tea extract)—200 mg
Taurine
Vitamin C—90 mg
Ginseng root extract
Acidifier
Sweetener
Thickener
Flavouring agent
Preservative

The total blend of caffeine, taurine and ginseng is present in an amount of 520 mg. The oral product has a volume of 60 mL per serving.

The oral product is prepared as set out in Example 1.

### Example 8—Results of Further Consumer Testing

A study was carried out to compare a product as prepared in Example 7 with a placebo (base formulation without actives). Blind testing was carried out where some test subjects consumed the shot of Example 7 while some were given the base formulation without actives. The consumers were subjected to cognitively demanding tasks and self-reported psychological instruments at various time-points post consumption of the product.

A cognitive battery administered via COMPASS (a computerized mental performance assessment system) included: Numeric Working Memory (NWM), Rapid Visual Information Processing (RVIP), Serial subtraction 7's, Serial subtraction 3's, and Corsi blocks. These tasks each evaluate a different aspect of cognitive function such as working memory, executive functions and sustained attention.

Self-reported measures on CompuSense (web-based software for consumer and sensory testing provided by Compusense Inc) included: Profile of Mood States (POMS), CATA (Check all that apply) questions, SAM (self-assessment manikin), and visual analogue scales (VAS, for: mental focus, distraction, alertness, boredom, physical jitteriness, relaxation, stimulation and concentration). All are validated self-reported questionnaires used to measure mood and emotions.

The Quality of Life Scale (QOLS) was used to assess the study population and bridge to other study populations. Again, it is a validated self-reported questionnaire used to measure anxiety in adults.

End of study interviews were employed for qualitative assessment.

### (a) Cognition

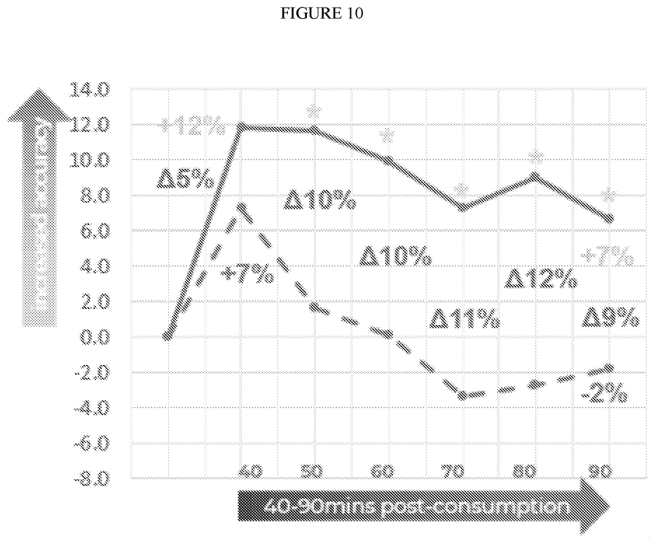

Cognitive accuracy was tested using Rapid Visual Information Processing (RVIP), which is the most demanding test within the cognitive battery. For RVIP, the results showed that the product of Example 7 outperformed the placebo, and maintained cognitive (RVIP) accuracy for up to 90 minutes post-consumption during challenging tasks. A significant change from placebo was shown at 50, 60, 70, 80 and 90 minutes post-consumption (this is shown with * in FIG. 10).

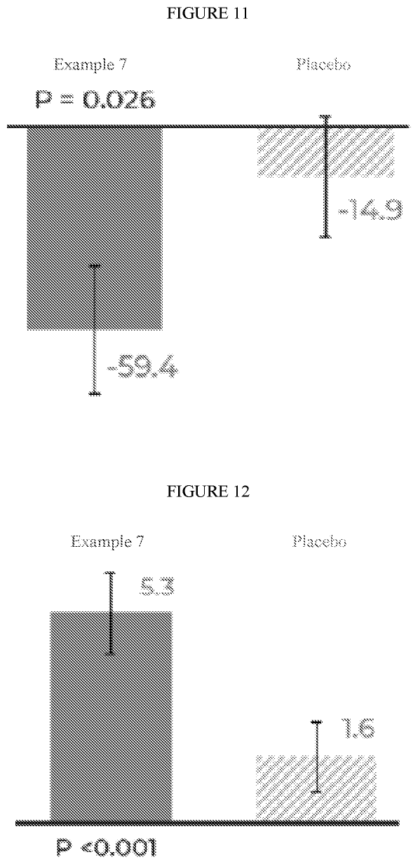

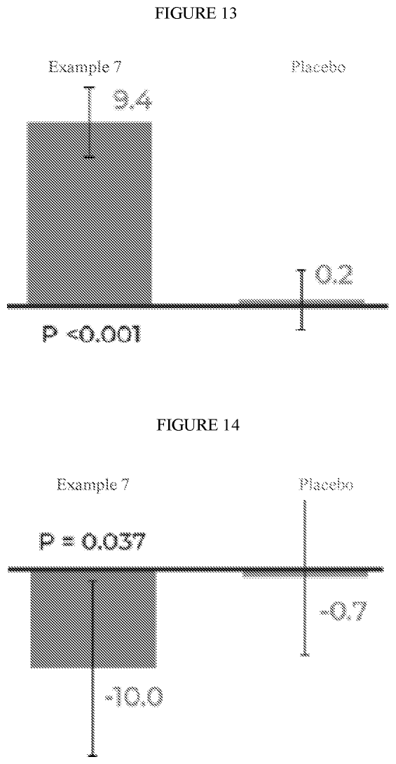

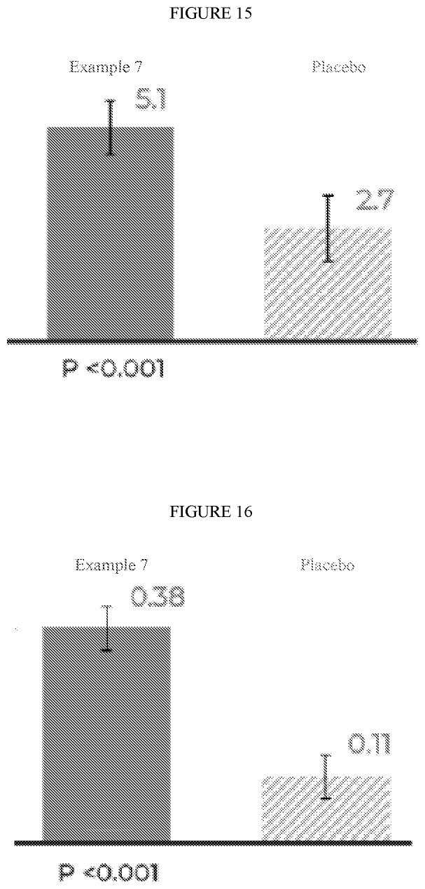

The following factors were also assessed for cognitive performance:
Memory recall—memory recall was tested using NWM in COMPASS. The results showed that the product of Example 7 achieved a statistically significant (p<0.001) increase in cognitive performance over the placebo by this measure (this is shown in FIG. 11).
Mental-arithmetic—assessed via timed and scored serial subtraction −3's. The oral product of Example 7 achieved a statistically significant (p=0.026) increase in cognitive performance over the placebo by this measure (this is shown in FIG. 12).
Cognitive accuracy—assessed using RVIP. The results showed that the product of Example 7 achieved a statistically significant (p<0.001) increase in cognitive performance over the placebo by this measure (this is shown in FIG. 13).
Mental processing—assessed using RVIP. The results showed that the product of Example 7 achieved a statistically significant (p=0.037) increase in cognitive performance over the placebo by this measure (this is shown in FIG. 14).
Mental arithmetic assessed via timed and scored serial subtraction −7's. The oral product of Example 7 achieved a statistically significant (p<0.001) increase in cognitive performance over the placebo by this measure (this is shown in FIG. 15).

A global cognition z-score was calculated based on the above measures. A z-score is a standard score indicating how many standard deviations an individual value is away from the mean and allow the comparison of scores from different tests. Z-scores were first calculated based on the non-adjusted raw data for each test. The respective z-scores were then combined (e.g. for RVIP, serial subtraction −3's and −7's). Lastly, scores were computed for each time period and adjusted by baseline subtraction. The oral product of Example 7 achieved a statistically significant (p<0.001) increase in global cognitive performance over the placebo (this is shown in FIG. 16).

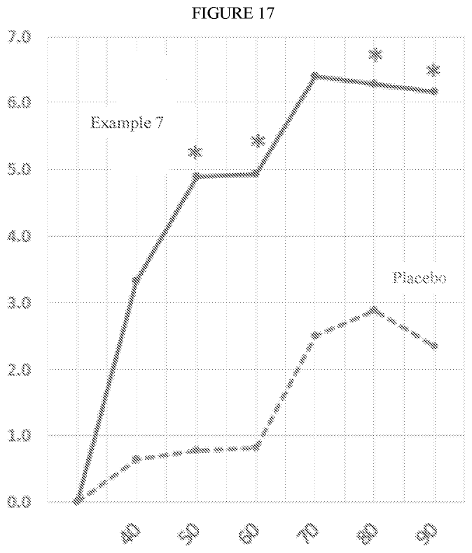

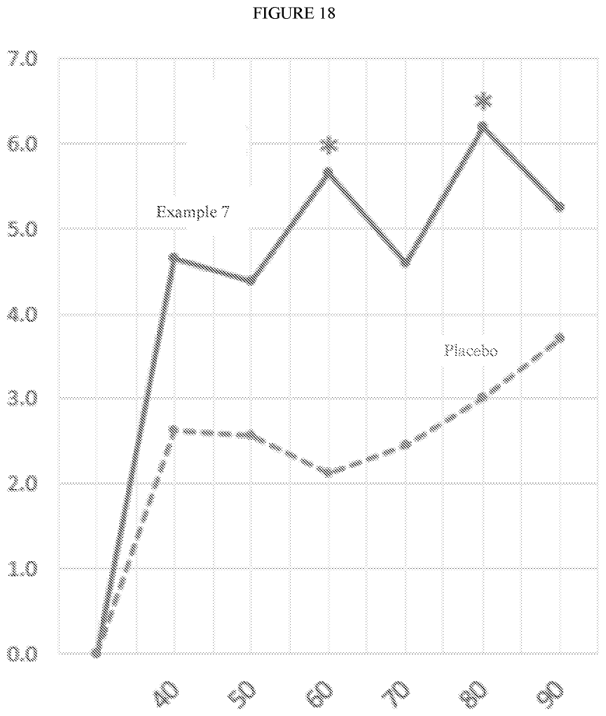

The product of Example 7 was also found to outperform the placebo for mental arithmetic and maintain performance for up to 90 minutes post-consumption as measured by serial subtraction −3's (see FIG. 17) and serial subtraction −7's (see FIG. 18). Statistically significant changes from placebo are shown in FIGS. 17 and 18 with *.

### (b) Perceived Mood State

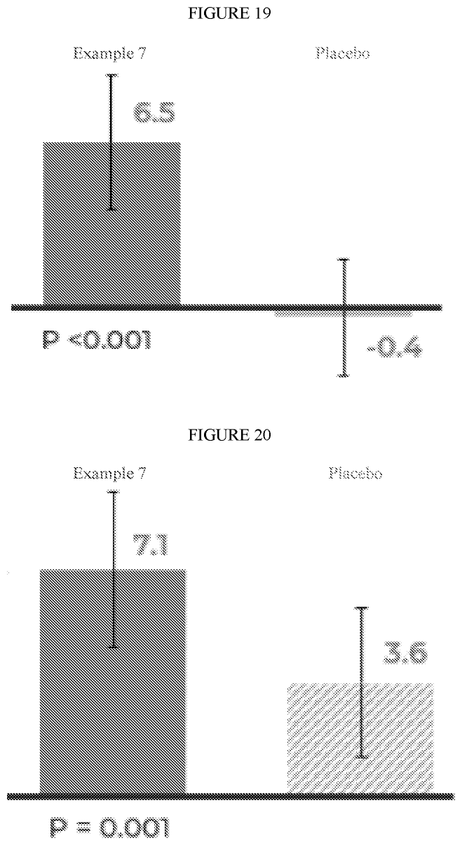

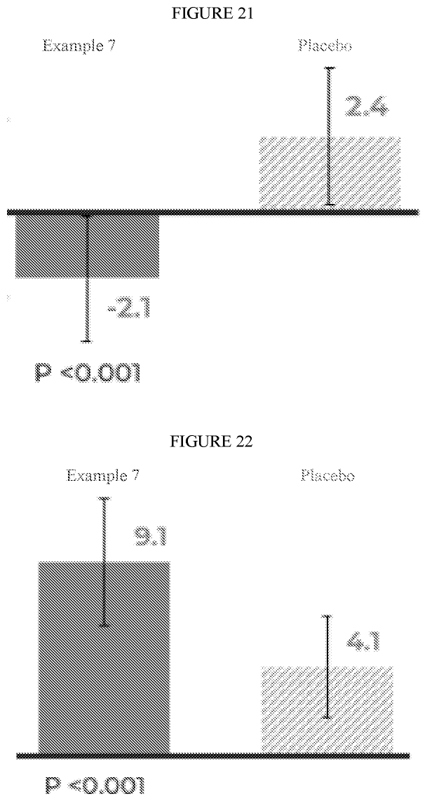

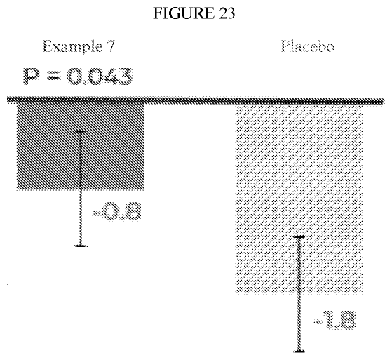

The following factors were assessed:
Alertness—consumers were tested using VAS. The results showed that the oral product of Example 7 achieved a statistically significant (p<0.001) increase in performance over placebo by this measure. This is shown in FIG. 19.
Physical energy—consumers were tested using VAS. The results showed that the oral product of Example 7 achieved a statistically significant (p=0.001) increase in performance over placebo by this measure, based on the average score taken over all post-consumption time points between 40-90 minutes. This is shown in FIG. 20.
Fatigue reduction—consumers were tested using VAS. The results showed that the oral product of Example 7 achieved a statistically significant (p=0.001) increase in performance over placebo by this measure. This is shown in FIG. 21.
Feeling of stimulation—consumers were tested using VAS. The results showed that the oral product of Example 7 achieved a statistically significant (p<0.001) increase in performance over placebo by this measure, based on the average score taken over all post-consumption time points between 40-90 minutes. This is shown in FIG. 22.
Vigour—reduced vigour was tested using VAS. The results showed that the oral product of Example 7 achieved a statistically significant (p<0.001) increase in performance over placebo (i.e. a lesser reduction in vigour over placebo) by this measure. This is shown in FIG. 23.

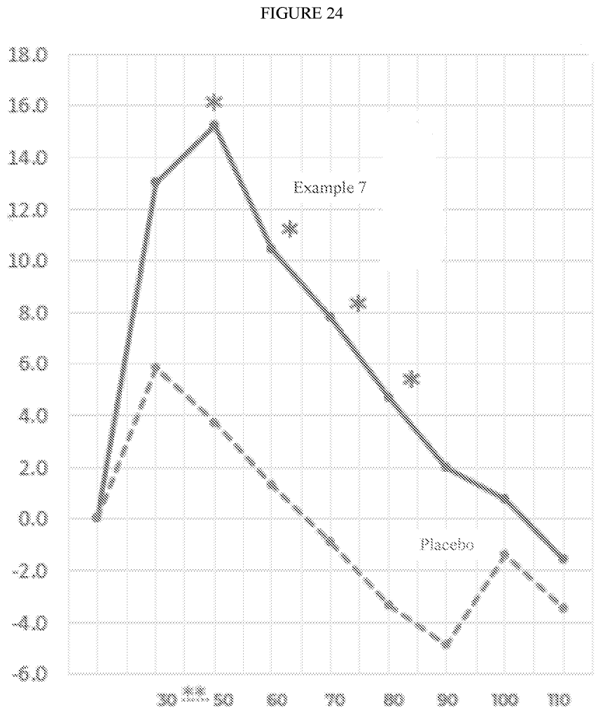

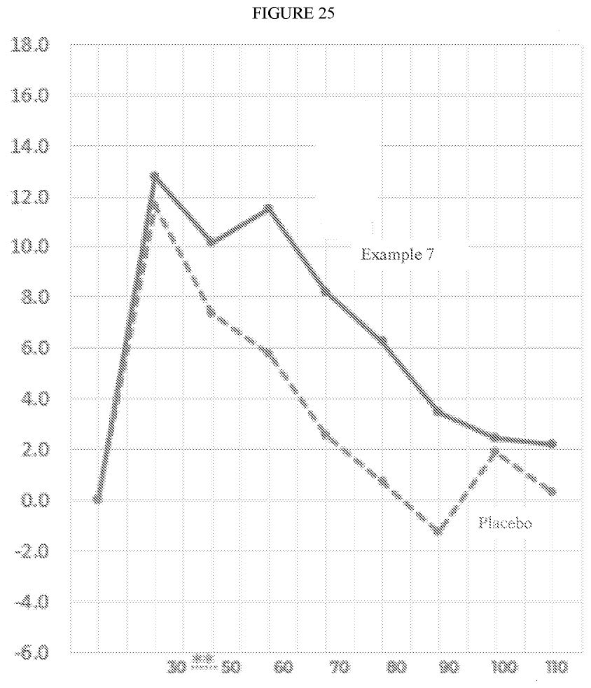

The effects of the oral product of Example 7 were also observed to be sustained for up to 90 minutes post-consumption. This is shown in FIG. 24 for perceived alertness over time and in FIG. 25 for perceived physical energy over time, each measured by VAS. Statistically significant changes from placebo are shown in FIGS. 24 and 25 with *.

The participants in the study (n=37, across groups) were aged between 19 and 30 years old and were considered to be healthy adults. Participants familiarised themselves with the cognitive batter tests for 1 week prior to consumption and refrained from physical activity and the consumption of caffeine 10 hours prior to the baseline testing, and any food and drink 1 hour before testing.

Cognitive battery tests were carried out before administration of any product (VAS, POMS, CATA, NWM, Corsi blocks, RVIP, serial −7's substraction and serial −3's subtraction). These tests provide a baseline cognitive battery.

Each participant then consumed the oral products as specified; the products were tested in a double-blind approach. Around 30 minutes after consumption, the participants were subjected to the same cognitive and mood battery tests. These tests were repeated at 10 minute intervals.

The results of the study are shown in FIGS. 10 to 25.

The results show how the oral product of the present invention had an improved performance in multiple cognitive domains versus placebo: faster memory recall, increased cognitive accuracy, greater mental processing, and improved mental arithmetic. Statistically significant improvements were also observed in multiple perceived effects versus placebo including heightened alertness, more physical energy, reduced fatigue, and greater feelings of stimulation. The qualitative post-study interviews confirmed the quantitative measures.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

### Numbered Clauses

1. An oral product comprising a combination of active ingredients, wherein the combination comprises:
(i) caffeine;
(ii) a combination of B vitamins comprising at least vitamin B2, vitamin B3, vitamin B6, vitamin B9 and vitamin B12; and
(iii) taurine.

2. An oral product according to clause 1, wherein the (ii) combination of B vitamins further comprises one or more of vitamin B1, vitamin B5 or vitamin B7.

3. An oral product according to clause 1 or 2, wherein the (ii) combination of B vitamins comprises a combination of vitamin B2, vitamin B3, vitamin B6, vitamin B9, vitamin B12, vitamin B1, vitamin B5 and vitamin B7.

4. An oral product according to any one of clauses 1 to 3, wherein the total amount of B vitamins included in the combination is from about 0.001% to about 1% by weight of the oral product.

5. An oral product according to any one of clauses 1 to 4, wherein the caffeine is present in an amount of from about 0.01% to about 5% by weight of the oral product.

6. An oral product according to any one of clauses 1 to 5, wherein the taurine is present in an amount of from about 0.001% to about 5% by weight of the oral product.

7. An oral product according to any one of clauses 1 to 6, wherein the caffeine and taurine are present in a weight ratio of from about 5:1 to about 1:1.

8. An oral product according to any one of clauses 1 to 7, wherein the caffeine and taurine are present in a weight ratio of about 1:1.

9. An oral product according to any one of clauses 1 to 8, wherein the caffeine and total amount of vitamin B are present in a weight ratio of from about 1:1 to about 15:1.

10. An oral product according to any one of clauses 1 to 9, wherein the taurine and total amount of vitamin B are present in a weight ratio of from about 10:1 to about 1:2.

11. An oral product according to any one of clauses 1 to 10, wherein the combination of active ingredients further comprises (iv) vitamin C, optionally wherein the vitamin C is present in an amount of from about 0.1% to about 5% by weight of the oral product.

12. An oral product according to clause 11, wherein the vitamin C and total amount of vitamin B are present in a weight ratio of from about 1:1 to about 20:1.

13. An oral product according to any one of clauses 1 to 12, wherein the combination of active ingredients further comprises (v) ginseng, optionally wherein the ginseng is present in an amount of from about 0.01% to about 5% by weight of the oral product.

14. An oral product according to any one of clauses 1 to 13, wherein the combination of active ingredients further comprises (vi) vitamin D.

15. An oral product according to any one of clauses 1 to 14, wherein the oral product comprises the combination of active ingredients in an amount of from about 0.5% to about 10% by weight of the oral product.

16. An oral product according to any one of clauses 1 to 15 further comprising one or more additives selected from the group consisting of a flavouring agent, sweetener, acidifying agent, thickener, filler, binder, humectant, preservative, and mixtures thereof.

17. An oral product according to clause 16, wherein the oral product comprises an acidifying agent.

18. An oral product according to any one of clauses 1 to 17, wherein the oral product further comprises zinc or zinc gluconate, optionally wherein the zinc or zinc gluconate is present in an amount of from about 0.001% to about 5% by weight of the oral product.

19. An oral product according to any one of clauses 1 to 18, wherein the oral product is an oral dosage form in the form of a solid, a gel or a liquid.

20. An oral product according to any one of clauses 1 to 19, wherein the oral product is in the form of a loose powder, lozenge, tablet, film or beverage.

21. An oral product according to any one of clauses 1 to 20, wherein the oral product is a liquid dosage form.

22. An oral product according to any one of clauses 1 to 21, wherein the liquid oral dosage form has a volume of from about 1 mL to about 200 ml of liquid, optionally wherein the liquid oral dosage form has a volume of from about 50 mL to about 100 ml of liquid.

23. An oral product according to clause 21 or 22, wherein the combination of active ingredients comprises:
(i) caffeine;
(ii) a combination of at least vitamin B2, vitamin B3, vitamin B6, vitamin B9 and vitamin B12;
(iii) taurine;
(iv) vitamin C; and
(v) ginseng.

24. An oral product according to any one of clauses 21 to 23, wherein the oral product further comprises water in an amount of from about 50% to about 99.9% by weight of the oral product.

25. An oral product according to any one of clauses 21 to 24, wherein the oral product comprises the combination of active ingredients in an amount of from about 0.5% to about 10% by weight of the oral product, and wherein the oral product comprises water in an amount of from about 90% to about 99.5% by weight of the oral product.

26. An oral product according to any one of clauses 21 to 25, wherein the liquid dosage form comprises:
(i) from about 50 mg to about 500 mg caffeine;
(ii) from about 1 mg to about 100 mg of a combination of B vitamins, where the combination comprises at least vitamin B2, vitamin B3, vitamin B6, vitamin B9 and vitamin B12; and
(iii) from about 1 mg to about 500 mg taurine.

27. An oral product according to clause 27, where in the liquid dosage form further comprises:
(iv) from about 50 mg to about 500 mg vitamin C; and/or
(v) from about 10 mg to about 300 mg ginseng.

28. A process for preparing an oral product as defined in any one of clauses 1 to 28, the process comprising:
(a) combining active ingredients, wherein the active ingredients include: (i) caffeine; (ii) a combination of B vitamins comprising at least vitamin B2, vitamin B3, vitamin B6, vitamin B9 and vitamin B12; and (iii) taurine,
(b) contacting the active ingredients with water, and
(c) mixing the active ingredients with water to prepare an oral product.

29. Use of a combination of active ingredients to increase alertness and/or energy levels of a human or animal, wherein the combination of active ingredients comprises (i) caffeine; (ii) a combination of B vitamins comprising at least vitamin B2, vitamin B3, vitamin B6, vitamin B9 and vitamin B12; and (iii) taurine.

30. An oral product comprising a combination of active ingredients, wherein the combination comprises:
(i) caffeine;
(ii) ginseng; and
(iii) taurine.

31. An oral product in liquid form comprising a combination of active ingredients, wherein the combination of active ingredients comprises:
(i) caffeine;
(ii) a combination of B vitamins comprising vitamin B2, vitamin B3, vitamin B6, vitamin B9 and vitamin B12; and
(iii) ginseng.

32. An oral product comprising a combination of active ingredients, wherein the combination of active ingredients comprises:
(i) caffeine;
(ii) at least one B vitamin selected from the group consisting of vitamin B1, vitamin B2, vitamin B3, vitamin B5, vitamin B6, vitamin B7, vitamin B9 and vitamin B12;
(iii) taurine; and
(iv) vitamin C.

1. An oral product comprising a combination of active ingredients, wherein the combination comprises:
(i) caffeine;
(ii) a combination of B vitamins comprising at least vitamin B1, vitamin B2, vitamin B6, and vitamin B7; and
(iii) taurine and/or ginseng; optionally wherein the caffeine: ginseng weight ratio is from 1:5 to 5:1.

2. An oral product according to claim 1, wherein the (ii) combination of B vitamins further comprises one or more of vitamin B3, vitamin B5, vitamin B9 or vitamin B12; and/or wherein the (ii) combination of B vitamins consists essentially of vitamin B1, vitamin B2, vitamin B6, and vitamin B7, preferably wherein the (ii) combination of B vitamins consists of vitamin B1, vitamin B2, vitamin B6 and vitamin B7.

3. (canceled)

4. An oral product according to claim 1, wherein the total amount of B vitamins included in the combination is from about 0.001% to about 1% by weight of the oral product.

5. An oral product according to claim 1, wherein the caffeine is present in an amount of from about 0.01% to about 5% by weight of the oral product.

6. An oral product according to claim 1, wherein the taurine is present in an amount of from about 0.001% to about 5% by weight of the oral product.

7. An oral product according to claim 1, wherein; the caffeine and taurine are present in a weight ratio of from about 5:1 to about 1:1, optionally wherein the caffeine and taurine are present in a weight ratio of about 1:1.

8. (canceled)

9. An oral product according to claim 1, wherein the caffeine and total amount of vitamin B are present in a weight ratio of from about 1:1 to about 30:1.

10. An oral product according to claim 1, wherein the taurine and total amount of vitamin B are present in a weight ratio of from about 10:1 to about 1:2.

11. An oral product according to claim 1, wherein the combination of active ingredients further comprises (iv) vitamin C, optionally wherein the vitamin C is present in an amount of from about 0.01% to about 5% by weight of the oral product; and/or wherein the combination of active ingredients further comprises (vi) vitamin D.

12. An oral product comprising a combination of active ingredients, wherein the combination comprises:
(i) caffeine;
(ii) ginseng; and
(iii) taurine; wherein the caffeine and taurine are present in a weight ratio of from about 5:1 to about 1:1; optionally wherein the caffeine: ginseng weight ratio is from 1:5 to 5:1.

13. An oral product according to claim 12, wherein:
the caffeine is present in an amount of from about 0.01% to about 5% by weight of the oral product;
the taurine is present in an amount of from about 0.001% to about 5% by weight of the oral product; and/or
the ginseng is present in an amount of from about 0.01% to about 5% by weight of the oral product.

14. An oral product according to claim 1, wherein the oral product comprises the combination of active ingredients in an amount of from about 0.5% to about 10% by weight of the oral product.

15. (canceled)

16. (canceled)

17. An oral product according to claim 1, wherein the oral product further comprises zinc or zinc gluconate, optionally wherein the zinc or zinc gluconate is present in an amount of from about 0.001% to about 5% by weight of the oral product.

18. An oral product according to claim 1, wherein the oral product is a liquid dosage form; optionally wherein the liquid oral dosage form has a volume of from about 1 mL to about 200 mL of liquid, optionally wherein the liquid oral dosage form has a volume of from about 50 mL to about 100 mL of liquid.

19. (canceled)

20. An oral product according to claim 12, wherein the oral product further comprises water in an amount of from about 50% to about 99.9% by weight of the oral product.

21. An oral product according to claim 20, wherein the liquid dosage form comprises:
(i) from about 50 mg to about 500 mg caffeine;
(ii) from about 1 mg to about 100 mg of a combination of B vitamins, where the combination comprises at least vitamin B1, vitamin B2, vitamin B6, and vitamin B7; and
(iii) from about 1 mg to about 500 mg taurine and/or from about 10 mg to about 300 mg ginseng;
optionally wherein the liquid dosage form further comprises:
(iv) from about 10 mg to about 500 mg vitamin C;
or
wherein the liquid dosage form comprises:
(i) from about 50 mg to about 500 mg caffeine;
(ii) from about 1 mg to about 500 mg taurine;
(iii) from about 10 mg to about 300 mg ginseng;
optionally wherein the liquid dosage form further comprises:
(iv) from about 10 mg to about 500 mg vitamin C.

22. A process for preparing an oral product as defined in claim 1, the process comprising:
(a) combining active ingredients, wherein the active ingredients include: (i) caffeine; (ii) a combination of B vitamins comprising at least vitamin B1, vitamin B2, vitamin B6, and vitamin B7; and (iii) taurine, taurine and/or ginseng; or wherein the active ingredients include: (i) caffeine; (ii) taurine; and (iii) ginseng;
(b) contacting the active ingredients with water, and
(c) mixing the active ingredients with water to prepare an oral product.

23. A composition comprising a combination of active ingredients to increase alertness and/or energy levels, or to increase the cognitive performance, of a human or animal, wherein the combination of active ingredients comprises (i) caffeine; (ii) a combination of B vitamins comprising at least vitamin B1, vitamin B2, vitamin B6, and vitamin B7; and (iii) taurine and/or ginseng; or wherein the combination of active ingredients comprises (i) caffeine; (ii) taurine; and (iii) ginseng.

24-56. (canceled)

57. An oral product according to claim 1, wherein the ginseng is present in an amount of from about 0.01% to about 5% by weight of the oral product.

58. An oral product according to claim 1, wherein the ginseng and total amount of vitamin B are present in a weight ratio of from about 1:1 to about 30:1.
